(19) United States
(12) Patent Application Publication
(10) Pub. No.: US 2011/0028371 A1
(43) Pub. Date: Feb. 3, 2011

(54) HYBRID COPOLYMERS

(75) Inventors: Klin A. RODRIGUES, Signal Mountain, TN (US); Allen M. Carrier, Hixson, TN (US); Jannifer Sanders, Hixson, TN (US)

Correspondence Address: AKZO NOBEL INC., LEGAL & IP, 120 WHITE PLAINS ROAD, SUITE 300, TARRYTOWN, NY, 10591, US

(73) Assignee: AKZO NOBEL N.V., Amhem (NL)

(21) Appl. No.: 12/533,802

(22) Filed: Jul. 31, 2009

(51) Int. Cl. C11D 3/39; C08F 20/00; C08F 251/00; C08F 251/02; C08F 255/00; C08G 63/91; G01N 33/44

(52) U.S. Cl. 510/220; 525/54.2; 525/54.23; 525/326.1; 510/276; 510/515; 510/109; 510/405; 510/513; 525/63; 436/85

(57) ABSTRACT

Hybrid copolymers include a synthetic polymer which is derived from at least one anionic or non-anionic ethylenically unsaturated monomer and a naturally derived hydroxyl containing chain transfer agent as an end group. The hybrid copolymer may be prepared as a blend with a builder or a chelating agent. Hybrid copolymers also include a synthetic portion derived from at least one ester monomer and a naturally derived hydroxyl containing chain transfer agent as an end group. Ester graft copolymers include a naturally derived hydroxyl moiety backbone and side chains derived from at least one ester monomer. Further, a method of determining the concentration of a hybrid copolymer in an aqueous system and methods of preparing a hybrid copolymer are also included.

### BACKGROUND

Hybrid polymers contain a portion of a naturally occurring oligomer or polymer and a moiety from a synthetically derived oligomer or polymer. One conventional method of making hybrid molecules utilizes water soluble monomers in the presence of an aqueous solution of a naturally derived, hydroxyl containing material as a chain transfer agent. Such a method is disclosed in US Patent application publication number US 2007-0021577 A1, which is wholly incorporated herein by reference.

### SUMMARY OF THE INVENTION

In an embodiment, the invention relates to a hybrid copolymer comprising a synthetic polymer derived from at least one anionic ethylenically unsaturated monomer and a naturally derived hydroxyl containing chain transfer agent as an end group. The chain transfer agent is present from about 75% by weight to about 99% by weight, based on the total weight of the hybrid copolymer.

In another embodiment, the invention is directed to a hybrid copolymer comprising a synthetic polymer derived from at least one of a non-anionic ethylenically unsaturated monomer and a naturally derived hydroxyl containing chain transfer agent as an end group.

In a further embodiment, the invention is directed to a hybrid copolymer comprising a synthetic portion derived from at least one ester monomer and a naturally derived hydroxyl containing chain transfer agent as an end group.

In another further embodiment, the invention relates to an ester graft copolymer comprising a naturally derived hydroxyl moiety backbone and side chains derived from at least one ester monomer.

In yet another embodiment, the invention is directed to a method of determining the concentration of a hybrid copolymer in an aqueous system. The method comprises reacting a sample of an aqueous hybrid copolymer comprising a synthetic polymer derived from at least one anionic ethylenically unsaturated monomer and a naturally derived hydroxyl containing chain transfer agent as the end group with an effective amount of photoactivator under conditions effective to cause the hybrid copolymer to absorb with the wavelength in the range of from 300 to 800 nanometers. The method further includes measuring the absorbance of the aqueous sample and comparing the absorbance of the aqueous sample to a predetermined calibration curve of known absorbances and concentrations. The method also includes comparing the absorbance of the aqueous sample to the known concentrations and known absorbences to determinine the concentration of the hybrid copolymer.

In still yet another embodiment, the invention relates to a method of preparing a hybrid copolymer. The method comprises polymerizing at least one monomer with a solution of a naturally derived hydroxyl containing chain transfer agent having a minor amount of secondary chain transfer agents.

In still yet another further embodiment, the invention is directed to a blend comprising a hybrid copolymer comprising a synthetic polymer derived from at least one anionic ethylenically unsaturated monomer and a naturally derived hydroxyl containing chain transfer agent as an end group and a builder or a chelating agent. The builder or chelating agent is selected from the group consisting of alkali metal or alkali-metal earth carbonates, alkali metal or alkali earth citrates, alkali metal or alkali earth silicates, glutamic acid N,N-diacetic acid (GLDA), methylglycine N,N-diacetic acid (MGDA) and combinations thereof.

### BRIEF DESCRIPTION OF THE DRAWING

The invention is best understood from the following detailed description when read in connection with the accompanying drawings. Included in the drawings are the following figures:

FIG. 1 is a graph depicting the results of dispersion tests conducted for 1 hour comparing a typical polyacrylate/maltodextrin blend with a hybrid copolymer containing greater than 75 weight percent maltodextrin as a chain transfer agent according to an embodiment of the invention.

FIG. 2 is a graph depicting the results of dispersion tests conducted for 24 hours comparing a typical polyacrylate/maltodextrin blend with a hybrid copolymer containing greater than 75 weight percent maltodextrin as a chain transfer agent according to an embodiment of the invention.

FIG. 3 is an illustration the results after a 1 hour dispersancy test between samples of polyacrylate having 0% maltodextrin, samples of a hybrid copolymer having at least one anionic ethylenically unsaturated monomer shown with various amounts of maltodextrin present, and a sample having 100% maltodextrin, after a 1 hour dispersancy test.

### DETAILED DESCRIPTION OF THE INVENTION

It has now been found that the hybrid copolymers according to the present invention can be prepared with a very high level of natural polymer transfer agent and still maintain the functionality of the synthetic polymers portion. In addition, new applications have been discovered for these hybrid polymers that were heretofore previously unknown.

As used herein, the term “hybrid copolymer” means a copolymer containing a synthetic polymer derived from an ethylenically unsaturated monomer that is chain terminated, or has an end group, with a naturally derived hydroxyl containing chain transfer agent. Optionally, in an embodiment of the present invention, the average molecular weight of the hybrid copolymer may be less than about 500,000. In further embodiments, the hybrid copolymer may be water soluble. For purposes of the present application, water soluble is defined as having a solubility of greater than about 1 gram of copolymer per 100 grams of water at 25° C.

The term “naturally derived hydroxyl containing chain transfer agent” as used herein, means a hydroxyl containing moiety obtained from plant sources directly or by enzymatic or fermentation processes. In an embodiment of the invention, these naturally derived hydroxyl containing chain transfer agents include, but are not limited, to small molecules such as glycerol, citric acid, lactic acid, tartaric acid, gluconic acid, ascorbic acid, glucoheptonic acid. The naturally derived hydroxyl containing chain transfer agents may also include saccharides or derivatives thereof. Suitable saccharides include, for example, monosaccharides and disaccharides such as sugars, as well as larger molecules such as oligosaccharides and polysaccharides (e.g., maltodextrins, pyrodextrins and starches). In an embodiment of the invention, when the naturally derived chain transfer agent is maltodextrin, pyrodextrin or a low molecular weight starch. It has been found that the chain transfer reaction does not work well when the chain transfer agent is not soluble in the system. For example, high molecular weight starches, such as those having molecular weights in the millions or those in granular form, are water dispersable and not water soluble. Accordingly, in embodiments of the invention, the average molecular weight of the chain transfer agent is preferably less than about 500,000. Starches having such exemplary molecular weights are water soluble. In another embodiment, the average molecular weight of the chain transfer agent may be less than about 100,000. In yet another embodiment, the average molecular weight of the chain transfer agent may be less than about 10,000. It has also been determined that for applications in which dispersancy and scale control is particularly desirable, a lower molecular weight, such as 10,000, of the chain transfer agent provides improved performance.

The naturally derived hydroxyl containing chain transfer agents also may include cellulose and its derivatives, as well as inulin and its derivatives, such as carboxymethyl inulin. Furthermore, these naturally derived hydroxyl containing chain transfer agents also include lignin and its derivatives, such as lignosulfonates. In an embodiment of the invention, lignin and its derivatives may be present in an amount of from about 0.1% to about 98% by weight, based on the total amount of the hybrid copolymer. In an embodiment of this invention the naturally derived chain transfer agents may be maltodextrins, pyrodextrins and chemically modified versions of maltodextrins and pyrodextrins. In another embodiment, the naturally derived chain transfer agent may be cellulose of inulin or chemically modified cellulose or inulin or a lignin derivative, such as lignosulfonate.

The naturally derived chain transfer agents can be used as obtained from their natural source or they can be chemically modified. Chemical modification includes hydrolysis by the action of acids, enzymes, oxidizers or heat, esterification or etherification. The modified naturally derived chain transfer agents, after undergoing chemical modification may be cationic, anionic, non-ionic or amphoteric or hydrophobically modified. In an embodiment of the invention, the hybrid copolymer may optionally be formed by polymerization catalyzed by, for example, a non-metal based radical initiator system.

In an aspect of the present invention, the invention relates to a hybrid copolymer that is anionic. In an embodiment according to this aspect, the anionic copolymer comprises a synthetic polymer produced from at least one anionic ethylenically unsaturated monomer that is chain terminated, or has an end group, with a naturally derived hydroxyl containing chain transfer agent.

As used herein, the term “anionic ethylenically unsaturated monomer” means an ethylenically unsaturated monomer which is capable of introducing a negative charge to the anionic hybrid copolymer. These anionic ethylenically unsaturated monomers can include, but are not limited to, acrylic acid, methacrylic acid, ethacrylic acid, α-chloro-acrylic acid, α-cyano acrylic acid, β-methyl-acrylic acid (crotonic acid), α-phenyl acrylic acid, β-acryloxy propionic acid, sorbic acid, α-chloro sorbic acid, angelic acid, cinnamic acid, p-chloro cinnamic acid, β-styryl acrylic acid (1-carboxy-4-phenyl butadiene-1,3), itaconic acid, maleic acid, citraconic acid, mesaconic acid, glutaconic acid, aconitic acid, fumaric acid, tricarboxy ethylene, muconic acid, 2-acryloxypropionic acid, 2-acrylamido-2-methyl propane sulfonic acid, vinyl sulfonic acid, sodium methallyl sulfonate, sulfonated styrene, allyloxybenzene sulfonic acid, vinyl phosphonic acid and maleic acid. Moieties such as maleic anhydride or acrylamide that can be derivatized (hydrolyzed) to moieties with a negative charge are also suitable. Combinations of anionic ethylenically unsaturated monomers can also be used. In an embodiment of the invention, the anionic ethylenically unsaturated monomer may preferably be acrylic acid, maleic acid, methacrylic acid, 2-acrylamido-2-methyl propane sulfonic acid or mixtures thereof

Based on the understanding of one of ordinary skill in the art, one would expect that the performance of the inventive anionic hybrid copolymers would decrease as the weight percent of the chain transfer agent in the polymer increases. For example, polysaccharides have little to no performance as dispersants by themselves. Surprisingly, however, it has been found that when the chain transfer agent content of the polymer is greater than 75 weight percent, performance is still maintained. For example the dispersancy performance of the anionic hybrid polymers is unexpectedly good even when using high amounts, such as 80, 90, 95 or even 99 and 99.5 weight percent, of the polysaccharide as a chain transfer agent.

Accordingly, the anionic hybrid copolymer further comprises a naturally derived hydroxyl containing chain transfer agent as the terminating moiety, or end group. In embodiments of the invention, the chain transfer agent may optionally be present from about 75% by weight to about 98% and most preferably from about 80 to about 99% by weight, based on the total weight of the anionic hybrid copolymer.

The anionic hybrid copolymer can be used as a constituent of a composition for a number of different applications including, but not limited to, cleaning, laundry, automatic dish washing (ADW), superabsorbent, fiberglass binder, rheology modifier, oil field, water treatment, dispersant, cementing and concrete compositions. For cleaning applications, the compositions may include, but are not limited to, detergent, fabric cleaner, automatic dishwashing detergent, rinse aids, glass cleaner, fabric care formulation, fabric softener, flocculants, coagulants, emulsion breakers, alkaline and acidic hard surface cleaners, laundry detergents and others. The compositions can also be used to clean surfaces in industrial and institutional cleaning applications. In an exemplary embodiment for automatic dishwashing detergent formulations, such formulations include phosphate, low phosphate and “zero” phosphate built formulations, in which the detergent is substantially free of phosphates. As used herein, low phosphate means less than 1500 ppm phosphate in the wash, in another embodiment less than about 1000 ppm phosphate in the wash, and in still another embodiment less that 500 ppm phosphate in the wash.

The anionic hybrid copolymers can also be used as scale control agents in cleaning, laundry, ADW, oil field, water treatment, and in any other aqueous system where scale buildup is an issue. The scales controlled include, but are not limited to, carbonate, sulfate, phosphate or silicate based scales such as calcium sulfate, barium sulfate, calcium ortho and polyphosphate, tripolyphosphate, magnesium carbonate, magnesium silicate and others.

In further embodiments, the anionic hybrid copolymers can also be used as dispersants in cleaning, oil field and water treatment applications, paint and coatings, paper coatings and other applications. These anionic hybrid copolymers can be used to disperse particulates including, but not limited to, minerals, clays, salts, metallic ores, metallic oxides, dirt, soils, talc, pigments, titanium dioxide, mica, silica, silicates, carbon black, iron oxide, kaolin clay, calcium carbonate, synthetic calcium carbonates, precipitated calcium carbonate, ground calcium carbonate, precipitated silica, kaolin clay or combinations thereof.

As used herein, the term “anionic hybrid copolymer adjunct ingredient” means ingredients that are typically used in formulations including the anionic hybrid copolymer. These anionic hybrid copolymer adjunct ingredients include, but are not limited to, water, surfactants, builders, phosphates, sodium carbonate, citrates, enzymes, buffers, perfumes, anti-foam agents, ion exchangers, alkalis, anti-redeposition materials, optical brighteners, fragrances, dyes, fillers, chelating agents, fabric whiteners, brighteners, sudsing control agents, solvents, hydrotropes, bleaching agents, bleach precursors, buffering agents, soil removal agents, soil release agents, fabric softening agent, opacifiers, water treatment chemicals, corrosion inhibitors, orthophosphates, zinc compounds, tolyltriazole, minerals, clays, salts, metallic ores, metallic oxides, talc, pigments, titanium dioxide, mica, silica, silicates, carbon black, iron oxide, kaolin clay, modified kaolin clays, calcium carbonate, synthetic calcium carbonates, fiberglass, cement and aluminum oxide. The surfactants can be anionic, non-ionic, such as low foaming non-ionic surfactants, cationic or zwitterionic. In an embodiment of the invention, the chelants may be glutamic acid N,N-diacetic acid (GLDA) and methylglycine N,N-diacetic acid (MGDA) and others.

Some other oil field uses for the anionic hybrid polymers of this invention include additives in cementing, drilling muds, dispersancy and spacer fluid applications. Often, the water encountered in oil field applications is sea water or brines from the formation. The water encountered in the oilfield can be very brackish. Hence, the polymers may also desirably be soluble in many brines and brackish waters. These brines may be sea water which contains about 3.5 percent NaC1 by weight or more severe brines that contain, for example, up to 3.5% KCl, up to 25% NaCl and up to 20% CaCl₂. Therefore, the polymers should be soluble in these systems for them to be effective as, for example, scale inhibitors. It has further been found that the higher the solubility of the anionic hybrid polymers in the brine, the higher the compatibility. The composition of synthetic seawater, moderate and severe calcium brines which are typical brines encountered in the oilfield is listed in Table 1 below.

TABLE 1
Typical brines encountered in the oilfield.
Brine preparation

| | | grams per liter | | | ppm | | |
|---|---|---|---|---|---|---|---|
| Brine number and description | | NaCl | CaCl₂•2H₂O | MgCl₂•6H₂O | Na | Ca | Mg |
| 1 | synthetic seawater | 24.074 | 1.61 | 11.436 | 9471 | 439 | 1368 |
| 2 | moderate calcium brine | 63.53 | 9.19 | | 24992 | 2506 | 0 |
| 3 | severe calcium brine | 127.05 | 91.875 | | 49981 | 25053 | 0 |

As described in Table 1, sea water contains around 35 grams per liter of a mixture of salts. The moderate and severe calcium brines contain around 70 and 200 grams per liter of a mixture of salts respectively.

In oil field applications, the scale inhibitor may be injected or squeezed or may be added topside to the produced water. Accordingly, embodiments of the invention also include mixtures of the anionic hybrid copolymer and a carrier fluid. The carrier fluid may be water, glycol, alcohol or oil. Preferably, the carrier fluid is water or brines or methanol. Methanol is often used to prevent the formation of methane hydrate (also known as methane clathrate or methane ice) structures downhole. In another embodiment of this invention, the anionic hybrid polymers may be soluble in methanol. Thus, the scale inhibiting polymers can be introduced in to the well bore in the methanol line. This is particularly advantageous since there is fixed number of lines that run in to the wellbore and this combination eliminates the need for another line.

In embodiments of the invention, the hybrid copolymers are latently-detectable, which means that they will not be detectable in the visible light range until the hybrid copolymer is contacted with a photoactivator. As defined herein, the “photoactivator” is an appropriate reagent or reagents which, when present in effective amounts, will react with the hybrid copolymer, thereby converting the hybrid copolymer into a chemical species which strongly absorbs in the region from about 300 to about 800 nanometers when activated with, for example, sulfuric acid and phenol. In an embodiment of this invention, the activated hybrid copolymer will absorb in the region from about 400 to about 700 nanometers.

A latently detectable moiety of this invention will be formed from a naturally derived hydroxyl containing chain transfer agent especially when it is saccharide or polysaccharide moiety. The photoactivator may be the combination of sulfuric acid and phenol (see Dubois et al, Anal. Chem. 28 (1956) p. 350 and Example 1 of U.S. Pat. No. 5,654,198, which is incorporated in its entirety by reference herein). Polymers typically tagged with latently detectable moieties exhibit a drop in efficacy when compared to polymers without these groups. This is especially true when the weight percent of the latently detectable moiety is over 10 or 20 percent of the polymer. However, it has been found that the hybrid polymers of the present invention perform well even when containing 50 percent or more of the latently detectable moiety. Thus, the advantages of good performance and ready detectability are provided, which allow monitoring the system and controlling scale without over dosing the scale control polymer.

In another aspect, the present invention relates to hybrid copolymers that are non-anionic. A hybrid copolymer that is non-anionic, as used herein, is a copolymer that comprises a synthetic polymer produced from at least one cationic or at least one nonionic ethylenically unsaturated monomer that is chain terminated, or has an end group, with a naturally derived hydroxyl containing chain transfer agent. As used herein, the term “cationic ethylenically unsaturated monomer” means an ethylenically unsaturated monomer which is capable of introducing a positive charge to the non-anionic hybrid copolymer. In an embodiment of the present invention, the cationic ethylenically unsaturated monomer has at least one amine functionality. Cationic derivatives of these non-anionic hybrid copolymers may be formed by forming amine salts of all or a portion of the amine functionality, by quaternizing all or a portion of the amine functionality to form quaternary ammonium salts, or by oxidizing all or a portion of the amine functionality to form N-oxide groups.

As used herein, the term “amine salt” means the nitrogen atom of the amine functionality is covalently bonded to from one to three organic groups and is associated with an anion.

As used herein, the term “quaternary ammonium salt” means that a nitrogen atom of the amine functionality is covalently bonded to four organic groups and is associated with an anion. These cationic derivatives can be synthesized by functionalizing the monomer before polymerization or by functionalizing the polymer after polymerization. These cationic ethylenically unsaturated monomers include, but are not limited to, N,N dialkylaminoalkyl(meth)acrylate, N-alkylaminoalkyl(meth)acrylate, N,N dialkylaminoalkyl(meth)acrylamide and N-alkylaminoalkyl(meth)acrylamide, where the alkyl groups are independently C₁₋₁₈ cyclic compounds such as 1-vinyl imidazole and the like. Aromatic amine containing monomers such as vinyl pyridine may also be used. Furthermore, monomers such as vinyl formamide, vinyl acetamide and the like which generate amine moieties on hydrolysis may also be used. Preferably the cationic ethylenically unsaturated monomer is N,N-dimethylaminoethyl methacrylate, tert-butylaminoethylmethacrylate and N,N-dimethylaminopropyl methacrylamide.

Cationic ethylenically unsaturated monomers that may be used are the quarternized derivatives of the above monomers as well as diallyldimethylammonium chloride also known as dimethyldiallylammonium chloride, (meth)acrylamidopropyl trimethylammonium chloride, 2-(meth)acryloyloxy ethyl trimethyl ammonium chloride, 2-(meth)acryloyloxy ethyl trimethyl ammonium methyl sulfate, 2-(meth)acryloyloxyethyltrimethyl ammonium chloride, N,N-Dimethylaminoethyl (meth)acrylate methyl chloride quaternary, methacryloyloxy ethyl betaine as well as other betaines and sulfobetaines, 2-(meth)acryloyloxy ethyl dimethyl ammonium hydrochloride, 3-(meth)acryloyloxy ethyl dimethyl ammonium hydroacetate, 2-(meth)acryloyloxy ethyl dimethyl cetyl ammonium chloride, 2-(meth)acryloyloxy ethyl diphenyl ammonium chloride and others.

As used herein, the term “nonionic ethylenically unsaturated monomer” means an ethylenically unsaturated monomer which does not introduce a charge in to the non-anionic hybrid copolymer. These nonionic ethylenically unsaturated monomers include, but are not limited to, acrylamide, methacrylamide, N alkyl(meth)acrylamide, N,N dialkyl(meth)acrylamide such as N,N dimethylacrylamide, hydroxyalkyl(meth)acrylates, alkyl(meth)acrylates such as methylacrylate and methylmethacrylate, vinyl acetate, vinyl morpholine, vinyl pyrrolidone, vinyl caprolactum, ethoxylated alkyl, alkaryl or aryl monomers such as methoxypolyethylene glycol (meth)acrylate, allyl glycidyl ether, allyl alcohol, glycerol (meth)acrylate, monomers containing silane, silanol and siloxane functionalities and others. The nonionic ethylenically unsaturated monomer is preferably water soluble.

The cationic or non-ionic hybrid copolymer has a naturally derived hydroxyl containing chain transfer agent as the terminating moiety, or end group. This chain transfer agent is preferably present from about 0.1% by weight to about 98%, more preferably from about 10 to about 95% and most preferably from about 20 to about 75% by weight, based on the total weight of the cationic or non-ionic hybrid copolymer.

In exemplary embodiments, the non-anionic hybrid copolymers can be used in fabric softener compositions as well as fabric care compositions. Suitable fabric softener formulations contain fabric softener actives, water, surfactants, electrolyte, phase stabilizing polymers, perfume, nonionic surfactant, non-aqueous solvent, silicones, fatty acid, dye, preservatives, optical brighteners, antifoam agents, and mixtures thereof. These fabric softener actives include, but are not limited, to diester quaternary ammonium compounds such as ditallowoyloxyethyl dimethyl ammonium chloride, dihydrogenated-tallowoyloxyethyl dimethyl ammonium chloride, dicanola-oyloxyethyl dimethyl ammonium chloride, ditallow dimethyl ammonium chloride, triethanolamine ester quats such as di-(hydrogenated tallowoyloxyethyl)-N,N-methylhydroxyethylammonium methylsulfate and di-(oleoyloxyethyl)-N,N-methylhydroxyethylammonium methylsulfate as well as others such as tritallow methyl ammonium chloride, methyl bis(tallow amidoethyl)-2-hydroxyethyl ammonium methyl sulfate, methyl bis(hydrogenated tallow amidoethyl)-2-hydroxyethyl ammonium methyl sulfate, methyl bis (oleyl amidoethyl)-2-hydroxyethyl ammonium methyl sulfate, ditallowoyloxyethyl dimethyl ammonium methyl sulfate, dihydrogenated-tallowoyloxyethyl dimethyl ammonium chloride, dicanola-oyloxyethyl dimethyl ammonium chloride, N-tallowoyloxyethyl-N-tallowoylaminopropyl methyl amine, 1,2-bis(hardened tallowoyloxy)-3-trimethylammonium propane chloride, dihardened tallow dimethyl ammonium chloride and mixtures thereof.

The preferred actives are diester quaternary ammonium (DEQA) compounds which are typically made by reacting alkanolamines such as MDEA (methyldiethanolamine) and TEA (triethanolamine) with fatty acids. Some materials that typically result from such reactions include N,N-di(acyl-oxyethyl)-N,N-dimethylammonium chloride or N,N-di(acyloxyethyl)-N,N-methylhydroxyethylammonium methylsulfate wherein the acyl group is derived from animal fats, unsaturated, and polyunsaturated, fatty acids, e.g., oleic acid, and/or partially hydrogenated fatty acids, derived from vegetable oils and/or partially hydrogenated vegetable oils, such as, canola oil, safflower oil, peanut oil, sunflower oil, corn oil, soybean oil, tall oil, rice bran oil, and the like. Those skilled in the art will recognize that active softener materials made from such process can comprise a combination of mono-, di-, and tri-esters depending on the process and the starting materials.

As used herein, the term “fabric care formulations” include, but are not limited to, formulations used to treat fabric to improve fabric softness, shape retention, fabric elasticity, fabric tensile strength, fabric tear strength, fabric lubrication, fabric relaxation, durable press, wrinkle resistance, wrinkle reduction, ease of ironing, abrasion resistance, fabric smoothing, anti-felting, anti-pilling, crispness, appearance enhancement, appearance rejuvenation, color protection, color rejuvenation, anti-shrinkage, static reduction, water absorbency or repellency, stain repellency, refreshing, anti-microbial, odor resistance, and mixtures thereof. In addition to the non-anionic hybrid copolymers, the fabric care formulations may contain ingredients such as cationic surfactants, amphoteric surfactants, fabric softener actives, sucrose esters, softening agents, other fabric care agents, dispersing media, such as water, alcohols, diols; emulsifiers, perfumes, wetting agents, viscosity modifiers, pH buffers, antibacterial agents, antioxidants, radical scavengers, chelants, antifoaming agents, and mixtures thereof.

In further embodiments of the invention, the non-anionic hybrid copolymers can be used as flocculants and coagulants for sludge dewatering and water clarification in waste water treatment applications. Further, domestic and industrial sewage contains suspended matter which must be removed. The suspended particles are predominantly stabilized due to their net negative surface charge. The cationic hybrid polymers disrupt this negative charge and enable removal of the suspended solids from the water. In still further embodiments, the non-anionic hybrid copolymers function as emulsion breakers for oil in water emulsions. These are useful in waste water treatment applications to comply with the limitations of fats oil and greases in the discharge water. In addition, the non-anionic hybrid copolymers function as reverse emulsion breakers in the oil field. In this application, small amounts of oil droplets are removed from the water continuous phase before the water can be safely returned to the environment.

In an embodiment of the invention the hybrid polymers can be uniformly mixed or blended with builders or chelating agents and then processed into powders or granules. For example, compositions including the hybrid copolymers of the present invention may include alkali metal or alkali-metal earth carbonates, citrates or silicates as exemplary builders suitable for use in detergent formulations. The term alkali metals are defined as the Group IA elements, such as lithium, sodium and potassium, whereas the alkali-metal earth metals are the Group HA elements which include beryllium, magnesium and calcium.

Powders as used herein are defined as having an average particle size of less than about 300 microns, whereas granules are particles of an average size of greater than about 300 microns. By uniformly mixing or blending the hybrid copolymer with the builder or chelating agent, the particles or granules provide less hygroscopic properties and afford easier handling and free flowing powders. Free flowing as used in this application are powders or granules that do not clump or fuse together. In an embodiment of this invention, the hybrid polymer is an anionic hybrid copolymer. In another embodiment of this invention, the builders or chelating agents that can be blended with the hybrid copolymer are sodium or potassium carbonate, sodium or potassium silicate sodium or potassium citrate or glutamic acid N,N-diacetic acid (GLDA) or and methylglycine N,N-diacetic acid (MGDA).

The hybrid polymers can be used in cosmetic and personal care compositions. Hybrid polymers useful in cosmetic and personal care compositions include both anionic and non-anionic hybrid copolymers. Cosmetic and personal care compositions include, for example, skin lotions and creams, skin gels, serums and liquids, facial and body cleansing products, wipes, liquid and bar soap, color cosmetic formulations, make-ups, foundations, sun care products, sunscreens, sunless tanning formulations, shampoos, conditioners, hair color formulations, hair relaxers, products with AHA and BHA and hair fixatives such as sprays, gels, mousses, pomades, and waxes, including low VOC hair fixatives and sunscreens. These cosmetic and personal care compositions may be in any form, including with out limitation, emulsions, gels, liquids, sprays, solids, mousses, powders, wipes, or sticks.

The cosmetic and personal care compositions contain suitable “cosmetic and personal care actives”. Suitable cosmetic and personal care active agents include, for example, sunscreen agents or actives, aesthetic enhancers, conditioning agents, anti-acne agents, antimicrobial agents, anti-inflammatory agents, analgesics, anti-erythemal agents, antiruritic agents, antiedemal agents, antipsoriatic agents, antifungal agents, skin protectants, vitamins, antioxidants, scavengers, antiirritants, antibacterial agents, antiviral agents, antiaging agents, protoprotection agents, hair growth enhancers, hair growth inhibitors, hair removal agents, antidandruff agents, anti-seborrheic agents, exfoliating agents, wound healing agents, anti-ectoparacitic agents, sebum modulators, immunomodulators, hormones, botanicals, moisturizers, astringents, cleansers, sensates, antibiotics, anesthetics, steroids, tissue healing substances, tissue regenerating substances, hydroxyalkyl urea, amino acids, peptides, minerals, ceramides, biohyaluronic acids, vitamins, skin lightening agents, self tanning agents, coenzyme Q10, niacinimide, capcasin, caffeine, and any combination of any of the foregoing.

Suitable sunscreen agents or actives useful in the present invention include any particulate sunscreen active that absorbs, scatters, or blocks ultraviolet (UV) radiation, such as UV-A and UV-B. Non-limiting examples of suitable particulate sunscreen agents include clays, agars, guars, nanoparticles, native and modified starches, modified cellulosics, zinc oxide, and titanium dioxide and any combination of the foregoing. Modified starches include, for example, DRY-FLO®PC lubricant (aluminum starch octenylsuccinate), DRY-FLO®AF lubricant (corn starch modified), DRY-FLO® ELITE LL lubricant (aluminum starch octenylsuccinate (and) lauryl lysine), DRY-FLO® ELITE BN lubricant (aluminum starch octenylsuccinate (and) boron nitride), all commercially available from National Starch and Chemical Company.

The sunscreen agents may include those that form a physical and/or chemical barrier between the UV radiation and the surface to which they are applied. Non-limiting examples of suitable sunscreen agents include ethylhexyl methoxycinnamate (octinoxate), ethylhexyl salicylate (octisalate), butylmethoxydibenzoylmethane, methoxydibenzoylmethane, avobenzone, benzophenone-3 (oxybenzone), octocrylene, aminobenzoic acid, cinoxate, dioxybenzone, homosalate, methyl anthranilate, octocrylene, octisalate, oxybenzone, padimate O, phenylbenzimidazole sulfonic acid, sulisobenzone, trolamine salicylate and any combination of any of the foregoing

The cosmetic and personal care compositions can optionally include one or more aesthetic enhancers (i.e., a material that imparts desirable tactile, visual, taste and/or olfactory properties to the surface to which the composition is applied) and can be either hydrophilic or hydrophobic. Non-limiting examples of commercial aesthetic enhancers together with their INCI names that are optionally suitable for use in the present invention include PURITY®21C starch (zea maize (corn) starch) and TAPIOCA PURE (tapioca starch), as well as combinations thereof, that are available from the National Starch and Chemical Company.

Suitable conditioning agents include, but are not limited to, cyclomethicone; petrolatum; dimethicone; dimethiconol; silicone, such as cyclopentasiloxane and diisostearoyl trimethylolpropane siloxy silicate; sodium hyaluronate; isopropyl palmitate; soybean oil; linoleic acid; PPG-12/saturated methylene diphenyldiisocyanate copolymer; urea; amodimethicone; trideceth-12; cetrimonium chloride; diphenyl dimethicone; propylene glycol; glycerin; hydroxyalkyl urea; tocopherol; quaternary amines; and any combination thereof.

The cosmetic and personal care compositions can optionally include one or more adjuvants, such as pH adjusters, emollients, humectants, conditioning agents, moisturizers, chelating agents, propellants, rheology modifiers and emulsifiers such as gelling agents, colorants, fragrances, odor masking agents, UV stabilizer, preservatives, and any combination of any of the foregoing. Examples of pH adjusters include, but are not limited to, aminomethyl propanol, aminomethylpropane diol, triethanolamine, triethylamine, citric acid, sodium hydroxide, acetic acid, potassium hydroxide, lactic acid, and any combination thereof.

The cosmetic and personal care compositions may also contain preservatives. Suitable preservatives include, but are not limited to, chlorophenesin, sorbic acid, disodium ethylenedinitrilotetraacetate, phenoxyethanol, methylparaben, ethylparaben, propylparaben, phytic acid, imidazolidinyl urea, sodium dehydroacetate, benzyl alcohol, methylchloroisothiazolinone, methylisothiazolinone, and any combination thereof. In an embodiment of the invention, the cosmetic and personal care composition generally contains from about 0.001% to about 20% by weight of preservatives, based on 100% weight of total composition. In another embodiment, the composition contains from about 0.1% to about 10% by weight of preservatives, based on 100% weight of total composition.

The cosmetic and personal care compositions may optionally contain thickeners or gelling agents. Examples of such gelling agents include, but are not limited to, synthetic polymers such as the acrylic-based Carbopol® series of thickeners available from B. F. Goodrich, Cleveland, Ohio and associative thickeners such as Aculyn™, available from Rohm & Haas, Philadelphia, Pa. Other exemplary gelling agents include, cellulosic thickeners, such as derivatized hydroxyethyl cellulose and methyl cellulose, starch-based thickeners, such as acetylated starch, and naturally occurring gums, such as agar, algin, gum arabic, guar gum and xanthan gum. Thickeners and rheology modifiers may also include without limitation acrylates/steareth-20 itaconate copolymer, acrylates/ceteth-20 itaconate copolymer, potato starch modified, hydroxypropyl starch phosphate, acrylates/aminoacrylates/C10-30 alkyl PEG-20 itaconate copolymer, carbomer, acrylates/C10-30 alkyl acrylate crosspolymer, hydroxypropylcellulose, hydroxyethylcellulose, sodium carboxymethylcellulose, polyacrylamide (and) C13-14 isoparaffin (and) laureth-7, acrylamides copolymer (and) mineral oil (and) C13-14 isoparaffin (and) polysorbate 85, hydroxyethylacrylate/sodium acrylol dimethyltaurate copolymer, and hydroxyethylacrylate/sodium acrylol dimethyltaurate copolymer.

In an embodiment of the invention, the cosmetic and personal care composition is a hair cosmetic composition. Optional conventional additives may also be incorporated into the hair cosmetic compositions of this invention to provide certain modifying properties to the composition. Included among these additives are silicones and silicone derivatives; humectants; moisturizers; plasticizers, such as glycerine, glycol and phthalate esters and ethers; emollients, lubricants and penetrants, such as lanolin compounds; fragrances and perfumes; UV absorbers; dyes, pigments and other colorants; anticorrosion agents; antioxidants; detackifying agents; combing aids and conditioning agents; antistatic agents; neutralizers; glossifiers; preservatives; proteins, protein derivatives and amino acids; vitamins; emulsifiers; surfactants; viscosity modifiers, thickeners and rheology modifiers; gelling agents; opacifiers; stabilizers; sequestering agents; chelating agents; pearling agents; aesthetic enhancers; fatty acids, fatty alcohols and triglycerides; botanical extracts; film formers; and clarifying agents. These additives are present in small, effective amounts to accomplish their function, and generally will comprise from about 0.01 to about 10% by weight each, and from about 0.01 to about 20% by weight total, based on the weight of the composition.

The hair cosmetic composition may optionally be a mousse. For mousses, the solvent may be a lower (C₁₋₄) alcohol, particularly methanol, ethanol, propanol, isopropanol, or butanol, although any solvent known in the art may be used.

Optionally, an embodiment of the invention may also comprise a spray. For sprays propellants include any optional propellant(s). Such propellants include, without limitation, ethers, such as dimethyl ether; one or more lower boiling hydrocarbons such as C₃-C₆ straight and branched chain hydrocarbons, for example, propane, butane, and isobutane; halogenated hydrocarbons, such as, hydrofluorocarbons, for example, 1,1-difluoroethane and 1,1,1,2-tetrafluoroethane, present as a liquefied gas; and the compressed gases, for example, nitrogen, air and carbon dioxide.

In further embodiments of the present invention in which the hybrid copolymer is non-ionic, the ethylenically unsaturated monomer may optionally be derived from at least one ester monomer. Exemplary ester monomers include, but are not limited to, dicarboxylic acid. Suitable ester monomers derived from dicarboxylic acid include, but are not limited to, monomethylmaleate, dimethylmaleate, monomethylitaconate, dimethylitaconate, monoethylmaleate, diethylmaleate, monoethylitaconate, diethylitaconate, monobutylmaleate, dibutylmaleate, monobutylitaconate and dibutylitaconate.

In yet another embodiment, the present invention provides an ester graft copolymer comprising a naturally derived hydroxyl moiety backbone and side chains derived from at least one ester monomer. In an embodiment of the invention, the ester monomer is derived from a carboxylic acid. In a further embodiment, the at least one ester monomer may be chosen from monomethylmaleate, dimethylmaleate, monomethylitaconate, dimethylitaconate, monoethylmaleate, diethylmaleate, monoethylitaconate, diethylitaconate, monobutylmaleate, dibutylmaleate, monobutylitaconate and dibutylitaconate.

In a further aspect, the invention relates to a method of determining the concentration of a hybrid copolymer or ester graft copolymer in an aqueous system. In embodiments according to this aspect, the method comprises reacting a sample of an aqueous hybrid or ester graft copolymer with an effective amount of photoactivator under conditions effective to cause the hybrid copolymer to absorb with the wavelength in the range of from 300 to 800 nanometers.

In an embodiment, the aqueous hybrid copolymer may comprise a synthetic polymer derived from at least one anionic ethylenically unsaturated monomer and a naturally derived hydroxyl containing chain transfer agent as the end group. The method further includes measuring the absorbance of the aqueous sample and comparing the absorbance of the aqueous sample to a predetermined calibration curve of known absorbances and concentrations. By comparing the aqueous sample to the known concentrations and know absorbances of known hybrid or ester graft copolymers copolymers, the concentration of the aqueous sample is determined. Once the concentration of the hybrid or ester graft copolymer in the aqueous sample is determined, the amount of additional copolymer that would be needed to maintain the desired level of copolymer in the aqueous system can be adjusted accordingly. This eliminates overdosing the aqueous system with excess copolymer and minimizes waste and costs.

In yet another aspect, the invention relates to a method of preparing a hybrid copolymer. The method of preparing the hybrid copolymer comprises reacting at least one monomer with a solution of a naturally derived hydroxyl containing chain transfer agent that includes only minor amounts of secondary chain transfer agents, such as sodium hypophosphite. In an embodiment of the invention, the secondary chain transfer agent may be less than 20 weight percent of the hybrid polymer. In another embodiment, solution of the naturally derived hydroxyl containing chain transfer agent may be substantially free of secondary transfer agents. The method may further comprise catalyzing the polymerizing step with an initiator that is substantially free of a metal ion initiating system at a temperature sufficient to activate said initiator.

In still yet another aspect, the invention relates to a blend of the hybrid copolymer and a builder or a chelating agent. Exemplary chelating agents suitable for use in the present invention include, but are not limited to, alkali metal or alkali-metal earth carbonates, alkali metal or alkali earth citrates, alkali metal or alkali earth silicates, glutamic acid N,N-diacetic acid (GLDA), methylglycine N,N-diacetic acid (MGDA) and combinations thereof. In an embodiment according to the invention, the blend may be a particulate containing a uniform dispersion of the hybrid copolymer and the builder or chelating agent. The particulate may also be a powder or a granule. In a further embodiment, the chain transfer agent may be present from about 75% by weight to about 98% by weight, based on the total weight of the anionic hybrid copolymer.

### EXAMPLES

The following examples are intended to exemplify the present invention but are not intended to limit the scope of the invention in any way. The breadth and scope of the invention are to be limited solely by the claims appended hereto.

### Example 1

### Synthesis of Anionic Hybrid Copolymer with 80 Weight Percent Chain Transfer Agent

200 grams of maltose as a chain transfer agent (Cargill Sweet Satin Maltose 80% aqueous solution, available from Cargill Inc., Cedar Rapids, Iowa) was initially dissolved in 200 grams of water in a reactor and heated to 98° C. A monomer solution containing 40 grams of acrylic acid in 120 grams of water was subsequently added to the reactor over a period of 90 minutes. An initiator solution comprising of 6.6 grams of sodium persulfate in 40 grams of water was added to the reactor at the same time as the monomer solution over a period of 90 minutes. The reaction product was held at 98° C. for an additional 60 minutes. The polymer was then partially neutralized by adding 20 grams of a 50% solution of NaOH. The final product was a light yellow solution with 31% solids.

### Example 2

### Synthesis of Anionic Hybrid Copolymer with 95 Weight Percent Polysaccharide Functionality

190 grams of maltodextrin as a polysaccharide chain transfer agent (Cargill MD™ 01918 dextrin, spray-dried maltodextrin obtained by enzymatic conversion of common corn starch, available from Cargill Inc., Cedar Rapids, Iowa) was initially dissolved in 200 grams of water in a reactor and heated to 95° C. A monomer solution containing 10 grams of acrylic acid dissolved in 75 g of water was subsequently added to the reactor over a period of one hour. An initiator solution comprising of 0.5 grams of sodium persulfate in 25 grams of water was added to the reactor at the same time as the monomer solution but over a period of 1 hour and 10 minutes. The reaction product was held at 95° C. for an additional 30 minutes. The polymer was then partially neutralized by adding 5 grams of a 50% solution of NaOH dissolved in 40 grams of water.

### Example 3

### Synthesis of Anionic Hybrid Copolymer with 85 Weight Percent Maltose Functionality

213 grams of maltose as a chain transfer agent (Cargill Sweet Satin Maltose 80% aqueous solution, available from Cargill Inc., Cedar Rapids, Iowa) was initially dissolved in 180 grams of water in a reactor and heated to 98° C. A monomer solution containing 30 grams of acrylic acid in 60 grams of water was subsequently added to the reactor over a period of 90 minutes. An initiator solution comprising of 6.6 grams of sodium persulfate in 40 grams of water was added to the reactor at the same time as the monomer solution over a period of 90 minutes. The reaction product was held at 98° C. for an additional 60 minutes. The polymer was then partially neutralized by adding 15 grams of a 50% solution of NaOH and the final product was a clear amber colored solution.

### Example 4

### Synthesis of Anionic Hybrid Copolymer with 85 Weight Percent Polysaccharide Functionality

213 grams of maltodextrin as a polysaccharide chain transfer agent (Cargill Sweet Satin Maltose 80% aqueous solution, available from Cargill Inc., Cedar Rapids, Iowa) was initially dissolved in 180 grams of water in a reactor and heated to 98° C. A monomer solution containing 30 grams of acrylic acid dissolved in 100 grams of water was subsequently added to the reactor over a period of 90 minutes. An initiator solution comprising of 6.6 grams of sodium persulfate in 40 grams of water was added to the reactor at the same time as the monomer solution over a period of ninety minutes. The reaction product was held at 98° C. for an additional 60 minutes. The polymer was then neutralized by adding 15 grams of a 50% solution of NaOH and the final product was an amber colored solution.

### Example 5

### Synthesis of Anionic Hybrid Copolymer with 90 Weight Percent Maltose Functionality

225 grams of maltose as a chain transfer agent (Cargill Sweet Satin Maltose 80% aqueous solution, available from Cargill Inc., Cedar Rapids, Iowa) was initially dissolved in 200 grams of water in a reactor and heated to 98° C. A monomer solution containing 20 grams of acrylic acid in 115 grams of water was subsequently added to the reactor over a period of 90 minutes. An initiator solution comprising of 6.6 grams of sodium persulfate in 40 grams of water was added to the reactor at the same time as the monomer solution over a period of 90 minutes. The reaction product was held at 98° C. for an additional 60 minutes. The polymer was then partially neutralized by adding 10 grams of a 50% solution of NaOH and the final product was a clear yellow colored solution.

### Example 6

### Synthesis of Anionic Hybrid Copolymer with 80 Weight Percent Polysaccharide as a Chain Transfer Agent

160 grams of maltodextrin as a polysaccharide chain transfer agent (STAR-DRI 180 DE 18 spray-dried maltodextrin available from Tate and Lyle, Decatur, Illinois) was initially dissolved in 200 grams of water in a reactor and heated to 98° C. A monomer solution containing 40 grams of acrylic acid in 120 grams of water was subsequently added to the reactor over a period of 90 minutes. An initiator solution comprising of 6.6 grams of sodium persulfate in 40 grams of water was added to the reactor at the same time as the monomer solution over a period of 90 minutes. The reaction product was held at 98° C. for an additional 60 minutes. The polymer was then partially neutralized by adding 20 grams of a 50% solution of NaOH and the final product was a light yellow solution.

### Example 7

### Synthesis of Anionic Hybrid Copolymer with 90 Weight Percent Polysaccharide as a Chain Transfer Agent

180 grams of maltodextrin as a polysaccharide chain transfer agent (STAR-DRI 180 DE 18 spray-dried maltodextrin available from Tate and Lyle, Decatur, Illinois) was initially dissolved in 200 grams of water in a reactor and heated to 98° C. A monomer solution containing 20 grams of acrylic acid in 115 grams of water was subsequently added to the reactor over a period of 90 minutes. An initiator solution comprising of 6.6 grams of sodium persulfate in 40 grams of water was added to the reactor at the same time as the monomer solution over a period of 90 minutes. The reaction product was held at 98° C. for an additional 60 minutes. The polymer was then partially neutralized by adding 10 grams of a 50% solution of NaOH and the final product was a clear yellow colored solution.

### Example 8

### Dispersancy Evaluation of Anionic Hybrid Copolymers

The polymers of Example 6 and 7 were evaluated in a clay suspension/dispersancy test. A control without any polymer was also tested. These materials were compared against a sodium polyacrylate sample (NaPAA) (ALCOSPERSE® 602N, available from Akzo Nobel Surface Chemistry, Chattanooga, Tenn.). The samples were prepared by adding 2% clay (50:50 rose clay: spinks blend clay) to deionized water. The samples were then stirred on a magnetic stir plate for 20 minutes, after which 0.1% active polymer was added and the samples were stirred for one minute more. The suspensions were then poured into 100 ml graduated cylinders and allowed to rest for one hour. The dispersancy performance was then rated on a scale of 1 to 5 with 1 being no dispersancy and 5 being very good dispersancy. The results are shown in Table 1.

TABLE 1

| | Dispersancy |
|---|---|
| | Performance on |
| Polymer | a scale of 1 to 5 |
| None | 1 |
| Star-DRI 180 DE (maltodextrin from | 1 |
| examples 6 & 7, comparative example) | |
| Hybrid copolymer of Example 6 | 5 |
| Hybrid copolymer of Example 7 | 5 |
| Alcosperse 602N (Synthetic polymer, | 5 |
| comparative example) | |

These data in Table 1 above indicate that the polymers of this invention are excellent dispersants. This is especially notable since these polymers contain 80 and 90% polysaccharide but the polysaccharide by itself has no dispersancy performance. Furthermore, they are comparable in performance to synthetic polymers (e.g., sodium polyacrylate) typically used in this type of application.

### Example 9

### Synthesis of Anionic Hybrid Copolymer with 85 Weight Percent Natural Polysaccharide Functionality

170 grams of maltodextrin as a polysaccharide chain transfer agent (Cargill MD™ 01918 dextrin, spray-dried maltodextrin obtained by enzymatic conversion of common corn starch, available from Cargill Inc., Cedar Rapids, Iowa) was initially dissolved in 250 grams of water in a reactor and heated to 98° C. A monomer solution containing 30 grams of acrylic acid in 60 grams was subsequently added to the reactor over a period of 90 minutes. An initiator solution comprising of 6.6 grams of sodium persulfate in 40 grams of water was added to the reactor at the same time as the monomer solution over a period of 90 minutes. The reaction product was held at 98° C. for an additional 60 minutes. The polymer was then partially neutralized by adding 30 grams of a 50% solution of NaOH and the pH of this solution was 7.

### Example 10-15

The procedure of Example 9 above was repeated for Examples 10-15 in which different levels of sulfonated monomer in the form of sodium 2-acrylamido-2-methylpropane sulfonate (AMPS) were used, as identified in Table 2. This monomer is available as a 50% aqueous solution which was then mixed with the acrylic acid and this monomer mixture was fed in to the reactor over 90 minutes as described above.

TABLE 2

| | | | wt % |
|---|---|---|---|
| | wt % | | maltodextrin |
| | acrylic | wt % | (Cargill MD |
| Example | acid | AMPS | 01918) |
| 10 | 15 | 0 | 85 |
| 11 | 13 | 2 | 85 |
| 12 | 10 | 5 | 85 |
| 13 | 7.5 | 7.5 | 85 |
| 14 | 5 | 10 | 85 |
| 15 | 0 | 15 | 85 |

### Example 16

The polymers from Examples 9 to 15 were then tested in 3 different brines designated Brine 1, 2 and 3 respectively Table 3.

TABLE 3

| | | Brine 1 | | Brine 2 | | Brine 3 | |
|---|---|---|---|---|---|---|---|
| | Polymer | Observation after | | Observation after | | Observation after | |
| Polymer of | concentration | 0 h, | 24 h, | 0 h, | 24 h, | 0 h, | |
| Example | [ppm] | 21° C. | 90° C. | 21° C. | 90° C. | 21° C. | 24 h, 90° C. |
| Synthetic | 250 | Y | Y | Y | X | X | X |
| homopolymer | 5000 | Y | Y | Y | X | X | X |
| (Sodium | 100000 | Y | Y | Y | X | X | X |
| polyacrylate) | | | | | | | |
| at pH 5 | | | | | | | |
| 9 | 250 | Y | Y | Y | Y | Y | Y |
| | 5000 | Y | Y | Y | Y | X | X |
| | 100000 | Y | Y | Y | Y | X | X |
| 10 | 250 | Y | Y | Y | Y | Y | Y |
| | 5000 | Y | Y | Y | Y | Y | Y |
| | 100000 | Y | Y | Y | Y | Y | redisperable |
| | | | | | | | precipitate |
| 11 | 250 | Y | Y | Y | Y | Y | Y |
| | 5000 | Y | Y | Y | Y | Y | Y |
| | 100000 | Y | Y | Y | Y | Y | Y |
| 12 | 250 | Y | Y | Y | Y | Y | Y |
| | 5000 | Y | Y | Y | Y | Y | Y |
| | 100000 | Y | Y | Y | Y | Y | X |
| 13 | 250 | Y | Y | Y | Y | Y | Y |
| | 5000 | Y | Y | Y | Y | Y | Y |
| | 100000 | Y | Y | Y | Y | Y | X |
| 15 | 250 | Y | Y | Y | Y | Y | Y |
| | 5000 | Y | Y | Y | Y | Y | Y |
| | 100000 | Y | Y | Y | Y | Y | X |

Y Compatible, clear solution
Uniform haze Hazy solution, no precipitate settling
Redispersable minimal precipitate settles, but uniformly redisperses with minimal agitation
precipitate
X Precipitate formed, either crystalline fiber-like structures or gross powder-like
precipitate

The data indicate that the hybrid copolymers of this invention containing acrylic acid and sulfonate monomer and 85 weight percent polysaccharide chain transfer agent are brine compatible. However, a corresponding synthetic homopolymer is not brine compatible.

### Example 17

158 grams of maltodextrin as a polysaccharide chain transfer agent (Cargill MD™ 01960 DE 11, available from Cargill Inc., Cedar Rapids, Iowa) was initially dissolved in 155 grams of water in a reactor and heated to 85° C. A monomer solution containing 39 grams of acrylic acid and 27.5 grams of a 50% solution of sodium 2-acrylamido-2-methylpropane sulfonate was subsequently added to the reactor over a period of 180 minutes. An initiator solution comprising of 8.3 grams of sodium persulfate in 33 grams of water was added to the reactor over a period of 195 minutes. The reaction product was held at 85° C. for an additional 30 minutes. The polymer was then partially neutralized by adding 14.5 grams of a 50% solution of NaOH and 66 grams of water.

### Example 18

Brine compatibility of the sulfonated anionic hybrid polymer containing 75 weight percent maltodextrin of Example 17 and a commercial sulfonated synthetic polymer (Aquatreat 545, available from Alco Chemical which is a copolymer of acrylic acid and sodium 2-acrylamido-2-methylpropane sulfonate) was tested in Brine 3, the composition of which is listed in Table 4. The data shown for these compatibility tests are shown below.

TABLE 4

| | | | Brine 3 | | | |
|---|---|---|---|---|---|---|
| | | Polymer | Observation after | | | |
| | Natural | concentration | 0 h, | 1 h, | 2 h, | 24 h, |
| Inhibitor | component | [ppm] | 21° C. | 60° C. | 90° C. | 90° C. |
| Example 13 | | 5000 | Y | Y | Y | Y |
| | | 25000 | Y | Y | Y | Y |
| | | 100000 | Y | Y | Y | X |
| Aquatreat 545 | | 5000 | X | Uniform | Uniform haze | X |
| from Alco | | | | haze | | |
| Chemical | | 25000 | X | X | X | X |
| | | 100000 | X | X | Uniform haze | X |

Y Compatible, clear solution
Uniform haze Hazy solution, no precipitate settling
Redispersable minimal precipitate settles, but uniformly redisperses with minimal agitation
precipitate
X Precipitate formed, either crystalline fiber-like structures or gross powder-like
precipitate

These data indicate that the hybrid copolymers of this invention are extremely brine compatible whereas corresponding synthetic polymers are not.

### Example 19

### Synthesis of Hybrid Copolymer with Polysaccharide Chain Transfer Agent as Well as a Secondary Chain Transfer Agent

221 grams of maltodextrin as a polysaccharide chain transfer agent (STAR-DRI 100 DE 10 spray-dried maltodextrin available from Tate and Lyle, Decatur, Ill.) and 40 g of sodium hypophosphite dihydrate (approximately 7.7 weight % based on the total weight of the polymer) as a secondary chain transfer agent was dissolved in 350 grams of water in a reactor and heated to 75° C. A monomer solution containing 221 grams of acrylic acid was subsequently added to the reactor over a period of three hours. An initiator solution comprising of 11 grams of sodium persulfate in 80 grams of water was added to the reactor at the same time as the monomer solution over a period of 3.5 hours. The reaction product was held at 75° C. for an additional 60 minutes. The polymer was then partially neutralized by adding 80 grams of a 50% solution of NaOH and the final product was a clear yellow colored solution.

### Example 20

### Synthesis of Ester Hybrid Copolymer Containing an Ester Monomer Derived from a Dicarboxylic Acid

45.9 grams of monomethylmaleate (ester monomer) was dissolved in 388 grams of water. 15.3 grams of ammonium hydroxide was added and the mixture was heated to 87C. 85 grams of maltodextrin of DE 18(Cargill MD™ 01918, spray-dried maltodextrin obtained by enzymatic conversion of common corn starch, available from Cargill Inc., Cedar Rapids, Iowa) was added just before the monomer and initiator feeds were started. A monomer solution containing a mixture of 168 grams of acrylic acid and 41.0 grams of hydroxyethyl methacrylate was added to the reactor over a period of 5 hours. A first initiator solution comprising of 21 grams of erythorbic acid dissolved in 99 grams of water was added over a period of 5.5 hours. A second initiator solution comprising of 21 grams of a 70% solution of tertiary butyl hydroperoxide dissolved in 109 grams of water was added over a period of 5.5 hours. The reaction product was held at 87° C. for 30 minutes. The final product was a clear light amber solution and had 33.8% solids.

### Example 21

The ester hybrid copolymer of Example 20 was evaluated for barium sulfate inhibition using the procedure described below:

### Part 1: Solution Preparation

1. Prepare Synthetic North Sea seawater (SW) brine.
a. Add the following salts identified in Table 5 to a glass volumetric flask and bring to volume with DI water. Weigh all +/−0.01 grams.
b. Buffer SW by adding 1 drop of acetic acid then adjust the pH with saturated sodium acetate solution to reach pH 6.1. Record amount added.
c. Filter brine through 0.45 μm membrane filter under vacuum to remove any dust particles that may affect test reproducibility.

TABLE 5
SW

| | g/L | g/2L | g/3L | record actual |
|---|---|---|---|---|
| NaCl | 24.074 | 48.148 | 72.222 | |
| CaCl₂*2H₂O | 1.57 | 3.14 | 4.71 | |
| MgCl₂*6H₂O | 11.436 | 22.872 | 34.308 | |
| KCl | 0.877 | 1.754 | 2.631 | |
| Na₂SO₄ | 4.376 | 8.752 | 13.128 | |

grams sodium acetate added

NOTE:
Biological growth occurs in this solution due to sulfate content. Use within 1 week of making.

2. Preparation of a standardized Forties formation water (FW) brine.
a. Add the following salts identified in Table 6 to a glass volumetric flask and bring to volume with DI water. Weigh all +/−0.01 grams.
b. Buffer SW by adding 1 drop of acetic acid then adjust the pH with saturated sodium acetate solution to reach pH 6.1. Record amount added.
c. Filter brine through 0.45 μm membrane filter under vacuum to remove any dust particles that may affect test reproducibility.

TABLE 6
FW

| | g/L | g/2L | g/3L | record actual |
|---|---|---|---|---|
| NaCl | 74.167 | 148.334 | 222.501 | |
| CaCl₂*2H₂O | 10.304 | 20.608 | 30.912 | |
| MgCl₂*6H₂O | 4.213 | 8.426 | 12.639 | |
| KCl | 0.709 | 1.418 | 2.127 | |
| BaCl₂*2H₂O | 0.448 | 0.896 | 1.344 | |

grams sodium acetate added

3. Prepare a 1% (10,000 ppm) active polymer solution for each inhibitor to be tested.
a. Weigh indicated grams of polymer into a volumetric flask and bring to volume with buffered, filtered seawater.
Grams of polymer (g) required can be calculated by the formula below.
g=(V×C)/S where
V is volume in mL of volumetric flask
C is concentration of polymer required (as weight %)
S is solids (active) content (in weight %) of the polymer
Example: A polymer has a solids content of 35%. To create 100mL of a 1 wt% (10,000 ppm) solution:
g=(100×1)/35=2.857 g of polymer in 100 mL of seawater

4. Prepare a buffer solution.
a. Add 8.2 g anhydrous sodium acetate to 100 g of DI water

5. Prepare a quenching solution. Since barium sulfate forms readily on cooling, an effective dosage of scale inhibitor is required to prevent further precipitation after the test ends.
a. Add 9 g KCl to a 3 L volumetric flask. Dissolve with DI water.
b. Add 1 active wt % ALCOFLOW 615 (˜67.5 grams)

g=(3000×1)/44.4=67.57 g of polymer in 3000 mL
c. Bring to volume with DI water.

### Part 2: Test Setup

6. Label 40 mL glass vials with inhibitor name and concentration to be tested and number 1 through max 30 samples. The numbers will indicate the run order for the test.

7. Add 15 mL of DI water to each vial numbered 1-3. These will be used to make the totals.

8. Add 15 mL of SW to each vial numbered 4-30.

9. Label a second set of glass vials with “FW”.

10. Add 15 mL of FW to each vial.

11. Place FW and SW vials in incubator or oven, but do not heat.

### Part 3: Test Period

12. Turn on incubator and set to heat to 80C.

13. Prepare SW for test. To each SW vial numbered 7-30,
a. Add 0.3 mL of sodium acetate buffer solution.
b. Add the appropriate amount of scale inhibitor solution to give desired concentration for 30 mL of sample.
Microliters of inhibitor solution (μl) required can be calculated by the formula below.
μl=[(V₁×C₁)/C₂]×1000, where
V₁ is volume in mL of test sample (SW+FW)
C₁ is concentration of polymer desired (in ppm)
C₂ is concentration of active polymer in inhibitor solution
Example: Desired test concentration is 50 ppm in a 30mL sample size (SW+FW).
Using a 10,000 ppm (1%) polymer solution:

μl =[(30×50)/10,000]×1000=150 μl

14. To each SW vial numbered 1-6,
a. Add 0.3 mL of sodium acetate buffer solution.
b. Add an equivalent amount of water in place of the average amount of scale inhibitor solution used to prepare samples.
c. Vials 1-3 will be used to determine ppm Ba for totals.
d. Vials 4-6 will be used to determine ppm Ba for blanks.

15. Heat solutions for a minimum of 2 hours.

16. At the end of 2 hours take one “FW” vial and #1 labeled SW out of the incubator/oven.

17. Pour the contents of the “FW” vial into the treated SW.

18. Return sample 1 to incubator/oven.

19. Set a timer to begin counting up for 2 hours. (This time period is critical.)

20. When 1 minute has passed, take one “FW” vial and #2 labeled SW out of the incubator/oven.

21. Return sample 2 to incubator/oven.

22. Repeat steps 17-19 with remaining numbered vials, keeping an interval of 1 minute between samples, until each “FW” has been added to a numbered vial.

23. Label a set of test tubes with inhibitor information or run number. These will be used for filtration step.

24. Weigh 5 g +/−0.02 g of quenching solution into each vial.

### Part 4: Filtration

25. When the 2 hour period expires, take vial #1 out of the incubator/oven.

26. Filter˜5 g (record weight) into previously prepared vial containing quenching solution, ensuring that the labels on the vials match.
a. Place open vial containing quenching solution on balance.
b. Draw sample into a 5 mL luer-lok syringe.
c. Fit syringe with 0.45 μm membrane syringe filter.
d. Weigh 5 grams filtrate into vial. Record grams filtrate added (for ppm correction).

27. Repeat this process with each sample at 1 minute intervals, so that each sample has been under test conditions for exactly 2 hours.

### Part 5: ppm Determination

28. Concentration of barium should be determined by ICP. All samples should be run the day of the test.

29. Percent inhibition can be calculated by the following calculation:

% inhibition=((S*d)−B)/(T−B), where
S=ppm Ba in sample
d=dilution factor (grams filtrate +5 grams quenching solution)/grams filtrate
B=ppm Ba in blank
T=ppm Ba in total

### Additional Test Information:

### Sample Matrix:

TABLE 7

| | ppm in samples | |
|---|---|---|
| | as tested | ½ dilution |
| Na | 20037 | 10019 |
| Ca | 1619 | 809 |
| Mg | 936 | 468 |
| K | 416 | 208 |
| Ba | 126 | 63 |
| SO₄ | 1480 | 740 |
| Cl | 25142 | 12571 |

Materials needed:
calcium chloride dihydrate
sodium chloride
magnesium chloride hexahydrate
potassium chloride
barium chloride dihydrate
sodium sulfate
acetic acid
sodium acetate
polymers to be evaluated

### ALCOFLOW 615

Equipment needed:
Analytical balance
Sample vials

These data in Table 8 below indicate that the hybrid materials are excellent barium sulfate inhibitors and compares well in performance with a commercial synthetic polymer that is used for this purpose.

TABLE 8

| | | % BaSO₄ inhibition | | |
|---|---|---|---|---|
| | % | Polymer level | Polymer level | Polymer level |
| Polymer | solids | 10 ppm | 25 ppm | 50 ppm |
| Alcoflow | | | | 98.1 |
| 300¹ | | | | |
| Polymer of | | 30.4 | 75.9 | 93.4 |
| Example 19 | | | | |
| Polymer of | 33 | 76.9 | 98.7 | 96.7 |
| Example 20 | | | | |

¹ALCOFLOW ® 300 barium sulfate scale inhibition synthetic polymer available from AkzoNobel Surface Chemistry, Chattanooga, TN.

The polymers of examples 19 and 20, as identified in Table 9, were also tested for calcium carbonate inhibition using the NACE test.

TABLE 9

| | weight percent | Inhibitor | |
|---|---|---|---|
| | polysaccharide | dosage | % CaCO₃ |
| Polymer | in polymer | (ppm) | inhibition |
| polyacrylic acid | 0 | 1 | 30 |
| | | 3 | 96 |
| | | 5 | 88 |
| Maltodextrin DE 10 (used in | 100 | 5 | 0 |
| the synthesis of Example 19) | | | |
| Maltodextrin DE 18 (used in | 100 | 5 | 0 |
| the synthesis of Example 20) | | | |
| Polymer of Example 19 | 45 | 1 | 32 |
| | | 3 | 75 |
| | | 5 | 96 |
| Polymer of Example 20 | 50 | 1 | 22 |
| | | 3 | 87 |
| | | 5 | 96 |

These data indicate that even though the hybrid polymers have 45 to 50 percent of a polysaccharide chain transfer agent, these polymers perform similar to a synthetic polymer. This is noteworthy because the polysaccharide used in these examples does not have any inhibition performance, yet the performance of the hybrid polymers does not drop even when used at very low levels (typical end use levels for calcium carbonate inhibition polymer are 10 ppm).

The ester hybrid copolymer of Example 20 was also tested for detectability using the procedure of Example 1 of U.S. Pat. No. 5,654,198 and following the procedure of Dubois et al. (Anal Chem. 1956, 28, 350) as shown in Table 10.

TABLE 10

| | Polymer of Example 9 | From Example of U.S. Pat. No. |
|---|---|---|
| Polymer | Absorbance at 490 nm | 5,654,198 Monomer A |
| concentration | Using 5% phenol | Absorbance at 490 nm |
| ppm | in a 1 cm cell | Using 5% phenol in a 1 cm cell |
| 1000 | 1.4160 | 1.868 |
| 100 | 0.6069 | 0.221 |
| 10 | 0.1108 | <0.05 |
| 1 | 0.0570 | <0.05 |

The data indicate that the hybrid ester copolymer of Example 20 is significantly more detectable that the Monomer A tag of Example 1 U.S. Pat. No. 5,654,198. Furthermore, monomer A is only incorporated in to the scale control polymer at about 10 weight percent. Thus the detectability of the hybrid polymers of this invention are far superior to that of US U.S. Pat. No. 5,654,198.

### Example 22

The ester hybrid copolymer synthesized in Example 20 was tested for compatibility in ethylene glycol.

TABLE 11

| | Solubility of | Solubility of |
|---|---|---|
| | the polymer as | the polymer as |
| | a 1% solution | a 50% solution |
| | in ethylene | in ethylene |
| Polymer | glycol | glycol |
| Example 20 | Soluble | Soluble |

These data indicate that the ester hybrid copolymers of the invention are extremely soluble in ethylene glycol.

### Example 23

The ester hybrid copolymer of Example 20 was tested for compatibility in methanol at a series of concentrations.

| Concentration of Polymer of Example 20 | |
|---|---|
| in the test solution (weight percent) | Result |
| 1 | Soluble |
| 20 | Soluble |
| 50 | Soluble |
| 80 | Soluble |

These data indicate that the ester hybrid copolymer of Example 20 is extremely soluble in methanol.

### Example 24

An automatic zero phosphate dishwash formulation was formulated containing an ester hybrid copolymer with an ester monomer derived from a dicarboxylic acid (Formulation A) and an anionic hybrid copolymer containing a sulfonate monomer (Formulation B), as shown in Table 12.

TABLE 12

Formulation

| | A | B |
|---|---|---|
| | Weight | Weight |
| | percent | percent |
| tri-Sodium citrate-2 H₂O | 10-30 | 10-30 |
| glutamic acid N,N-diacetic acid | 15-35 | 15-35 |
| (GLDA) or methylglycine N,N- | | |
| diacetic acid (MGDA) | | |
| Sodium carbonate | 15-25 | 15-25 |
| Sodium percarbonate | 10 | 10 |
| Sodium disilicate | 5 | 5 |
| TAED | 5 | 5 |
| Protease (e.g. Ovozyme 64T) | 3 | 3 |
| Amylase (e.g. Stainzyme 12T) | 2 | 2 |
| Polymer of Example 20 | 1-10 | 1-10 |
| Polymer of Example 14 | | 1-10 |
| Synperonic 810 | 2.5 | 2.5 |
| Synperonic 850 | 2.5 | 2.5 |
| Sodium sulfate | Rest | Rest |

### Example 25

### Synthesis of Ester Graft Copolymer Containing an Ester Monomer Derived from a Dicarboxylic Acid

115.4 grams of monomethylmaleate (ester monomer) was dissolved in 244 grams of water. 55 grams of 50% sodium hydroxide solution, and 0.0244 g of ferrous ammonium sulfate was added and the mixture was heated to 95° C. 192 grams of maltodextrin (STAR-DRI 100 DE 10 spray-dried maltodextrin available from Tate and Lyle, Decatur, Ill.) was added just before the monomer and initiator feeds were started. A monomer solution containing 76.9 grams was added to the reactor over a period of 4 hours. An initiator solution containing 47.2 g or 35% hydrogen peroxide and 5.8 g of sodium persulfate dissolved in 20 grams of water was added over a period of 4 hours. The reaction product was held at numeral and 95° C. for 60 minutes. The final product was a clear light amber solution and had 45.2% solids.

### Example 26

### Synthesis of Ester Hybrid Copolymer Containing an Ester Monomer Derived from a Dicarboxylic Acid

34 grams of dimethylmaleate (ester monomer) is dissolved in 150 grams of water. 5.4 grams of ammonium hydroxide is added and the mixture is heated to 87C. 170 grams of maltodextrin of DE 18(Cargill MD™ 01918, spray-dried maltodextrin obtained by enzymatic conversion of common corn starch, available from Cargill Inc., Cedar Rapids, Iowa) is added just before the monomer and initiator feeds were started. A monomer solution containing a mixture of 132.6 grams of acrylic acid and 3.4 grams of hydroxyethyl methacrylate was added to the reactor over a period of 5 hours. A first initiator solution comprising of 21 grams of erythorbic acid dissolved in 99 grams of water was added over a period of 5.5 hours. A second initiator solution comprising of 21 grams of a 70% solution of tertiary butyl hydroperoxide dissolved in 109 grams of water was added over a period of 5.5 hours. The reaction product was held at 87° C. for 60 minutes. The final product was a clear light amber solution and had 42.0% solids.

### Example 27

### Synthesis of Ester Hybrid Copolymer Containing an Ester Monomer Derived from a Dicarboxylic Acid

102 grams of monomethylmaleate (ester monomer) was dissolved in 150 grams of water. 5.4 grams of ammonium hydroxide was added and the mixture was heated to 87C. 170 grams of maltodextrin of DE 18(Cargill MD™ 01918, spray-dried maltodextrin obtained by enzymatic conversion of common corn starch, available from Cargill Inc., Cedar Rapids, Iowa) was added just before the monomer and initiator feeds were started. A monomer solution containing a mixture of 64.6 grams of acrylic acid and 3.4 grams of hydroxypropyl methacrylate was added to the reactor over a period of 5 hours. A first initiator solution comprising of 21 grams of erythorbic acid dissolved in 99 grams of water was added over a period of 5.5 hours. A second initiator solution comprising of 21 grams of a 70% solution of tertiary butyl hydroperoxide dissolved in 109 grams of water was added over a period of 5.5 hours. The reaction product was held at 87° C. for 60 minutes. The final product was a clear light amber solution and had 41.5% solids.

### Example 28

### Automatic Zero Phosphate Dishwash Powder Formulation

| Ingredients | wt % |
|---|---|
| Sodium citrate | 30 |
| Sodium carbonate | 20 |
| Polymer of Example 1 or 13 | 1 to 10 |
| Sodium disilicate | 10 |
| Perborate monohydrate | 6 |
| Tetraacetylethylenediamine | 2 |
| Enzymes | 2 |
| Sodium sulfate | 10 |

### Example 29

### Anti-Redeposition

The anionic hybrid copolymers of this invention were tested for anti-redeposition properties in a generic powdered detergent formulation. The powdered detergent formulation was as follows:

| Ingredient | wt % |
|---|---|
| Neodol 25-7 | 10 |
| Sodium carbonate | 46 |
| Sodium silicate | 3 |
| Sodium sulfate | 40 |

The test was conducted in a full scale washing machine using 3 cotton and 3 polyester/cotton swatches. The soil used was 17.5 g rose clay, 17.5 g spinks blend clay and 6.9 g oil blend (75:25 vegetable/mineral). The test was conducted for 3 cycles using 100 g powder detergent per wash load. The polymers were dosed in at 1.0 weight % of the detergent. The wash conditions used a temperature of 33.9° C. (93° F.), 150 ppm hardness and a 10 minute wash cycle.

L (luminance) a (color component) b (color component) values before the first cycle and after the third cycle was measured as L₁, aₗ, b₁ and L₂, a₂, b₂, respectively, using a spectrophotometer. ΔE (color difference) values were then calculated using the equation below

ΔE=[(L₁−L₂)²+(a₁−a₂)²+(b₁−b₂)²]^{0.5}

The data shown in Table 14 indicate that the anionic hybrid polymers of this invention show anti-redeposition/soil suspension properties even at low concentrations in the wash liquor (a lower AE indicates better anti-redeposition properties).

TABLE 14
Effect on anti-redeposition/soil suspension

| | ΔE | |
|---|---|---|
| Sample | Cotton | Poly/cotton |
| Control (no polymer) | 6.81 | 4.44 |
| Polymer of Example 6 | 2.27 | 2.07 |
| Polymer of Example 7 | 2.74 | 2.41 |

### Example 30

### Synthesis of Non-Ionic Hybrid Copolymer with Polysaccharide Chain Transfer Agent

50 grams of maltodextrin as a polysaccharide chain transfer agent (STAR-DRI 180 DE 18 spray-dried maltodextrin available from Tate and Lyle, Decatur, Ill.) was dissolved in 150 grams of water in a reactor and heated to 75° C. A monomer solution containing 50 grams of hydroxyethylacrylate was subsequently added to the reactor over a period of 50 minutes. An initiator solution comprising of 2 grams of V-50 [2,2′-Azobis (2 amidino-propane) dihydrochloride azo initiator from Wako Pure Chemical Industries, Ltd., Richmond, Va.] in 30 grams of water was added to the reactor at the same time as the monomer solution over a period of 60 minutes. The reaction product was held at 75° C. for an additional 60 minutes. The final product was a clear almost water white solution.

### Example 31

### Synthesis of Non-Anionic Hybrid Copolymer

150 grams of maltodextrin as a polysaccharide chain transfer agent (Cargill MD™ 01918 dextrin, spray-dried maltodextrin obtained by enzymatic conversion of common corn starch, available from Cargill Inc., Cedar Rapids, Iowa) was initially dissolved in 200 grams of water in a reactor and 70 g of HCl (37%) was added and heated to 98° C. A monomer solution containing 109 grams of dimethyl aminoethyl methacrylate dissolved in 160 grams of water was subsequently added to the reactor over a period of 90 minutes. An initiator solution comprising of 6.6 grams of sodium persulfate in 40 grams of water was added to the reactor at the same time as the monomer solution over a period of 90 minutes. The reaction product was held at 98° C. for an additional 60 minutes. The reaction product was then neutralized by adding 14 grams of a 50% solution of NaOH and the final product was an amber colored solution.

### Example 32

### Synthesis of Non-Anionic Hybrid Copolymer

35 grams of Amioca Starch was dispersed in 88 grams of water in a reactor and heated to 52. The starch was depolymerized by addition of 1.07 grams of concentrated sulfuric acid (98%). The suspension was held at 52° C. for 1.5 hours. The reaction was then neutralized with 1.84 grams of 50% NaOH solution and the temperature was raised to 90° C. for 15 minutes. The starch gelatinizes and the viscosity increased during the process and a gel is formed. The viscosity dropped after the gelatinization was completed. The temperature was lowered to 72 to 75° C. A solution of 80.7 grams of dimethyl diallyl ammonium chloride (62% in water) was added to the reactor over a period of 30 minutes. An initiator solution comprising of 0.2 grams of sodium persulfate in 20 grams of water was added to the reactor at the same time as the monomer solution over a period of 35 minutes. The reaction product was held at 98° C. for an additional 2 hours. The final product was a slightly opaque yellow colored solution.

### Example 33

### Synthesis of Non-Anionic Hybrid Copolymer

35 grams of Amioca Starch was dispersed in 88 grams of water in a reactor and heated to 52. The starch was depolymerized by addition of 0.52 grams of concentrated sulfuric acid (98%). This is half the acid used in Example 32 and causes less depolymerization of the starch resulting in a higher molecular weight. Thus the molecular weight of the polysaccharide chain transfer agent can be controlled. The suspension was held at 52° C. for 1.5 hours. The reaction was then neutralized with 0.92 grams of 50% NaOH solution and the temperature was raised to 90° C. for 15 minutes. The starch gelatinizes and the viscosity increased during the process and a gel was formed. The viscosity dropped after the gelatinization was completed. The reaction was diluted with 30 grams of water and the temperature was lowered to 72 to 75° C. A solution of 80.7 grams of dimethyl diallyl ammonium chloride (62% in water) was added to the reactor over a period of 30 minutes. An initiator solution comprising of 0.2 grams of sodium persulfate in 20 grams of water was added to the reactor at the same time as the monomer solution over a period of 35 minutes. The reaction product was held at 98° C. for an additional 2 hours. The final product was a clear light yellow colored solution.

### Example 34

### Synthesis of Non-Ionic Hybrid Copolymer with Polysaccharide (Inulin) Chain Transfer Agent

50 grams of a polysaccharide chain transfer agent (DEQUEST® PB11620 carboxymethyl inulin 20% solution available from Thermphos) was dissolved in 150 grams of water in a reactor and heated to 75° C. A monomer solution containing 50 grams of N,N dimethyl acrylamide was subsequently added to the reactor over a period of 50 minutes. An initiator solution comprising of 2 grams of V-50 [2,2′-azobis(2-amidinopropane) dihydrochloride] azo initiator from Wako Pure Chemical Industries, Ltd., Richmond, Va.] in 30 grams of water was added to the reactor at the same time as the monomer solution over a period of 60 minutes. The reaction product was held at 75° C. for an additional 60 minutes. The reaction product was diluted with 140 grams of water and the final product was a clear homogenous amber colored solution.

### Example 35

### Synthesis of Non-Anionic Hybrid Copolymer with Polysaccharide (Cellulosic) Chain Transfer Agent

Carboxymethyl cellulose (AQUALON® CMC 9M3ICT available from Hercules, Inc., Wilmington, Del.) was depolymerized in the following manner. Thirty grams of AQUALON® CMC was introduced in to 270 g of deionized water with stirring. 0.03 g of Ferrous ammonium sulfate hexahydrate and 2 g of hydrogen peroxide (H₂O₂) solution (35% active) was added. The mixture was heated to 60° C. and held at that temperature for 30 minutes. This depolymerized CMC solution was then heated to 90° C.

A monomer solution containing 50 grams of acrylamide (50% solution) is subsequently added to the reactor over a period of 50 minutes. An initiator solution comprising of 2 grams of V-086 2,2′-Azobis [2-methyl-N-(2-hydroxyethyl) propionamide] azo initiator from Wako Pure Chemical Industries, Ltd., Richmond, Va.] in 30 grams of water is added to the reactor at the same time as the monomer solution over a period of 60 minutes. The reaction product is held at 90° C. for an additional 60 minutes.

### Example 36

Typical dilute fabric softener formulations using non-anionic hybrid copolymers are listed below.

TABLE 15
Formulations of Dilute Traditional Softeners

| | |
|---|---|
| Ingredient | (%) |

Formula A

| | |
|---|---|
| distearyldimethylammonium Chloride | 6-9 |
| (75% active) | |
| Polymer of Example 32 | 0.1-3.0 |
| Perfume | 0.2-0.5 |
| Colorant | 0.001 |
| Water | Balance |

Formula B

| | |
|---|---|
| Quaternary dialkylimidazolines (75% | 6-9 |
| active) | |
| Polymer of Example 32 | 0.1-3.0 |
| Perfume | 0.2-0.5 |
| Colorant | 0.001 |
| Preservative | + |

### Example 37

Concentrated fabric softener compositions with non-anionic hybrid polymers are exemplified in Table 16.

TABLE 16
Ready-to-Use Rinse Conditioners at Triple Concentration
Formula C

| Ingredient | (%) |
|---|---|
| distearyldimethylammonium chloride | 14 |
| 75% | |
| Polymer of Example 33 | 3-10 |
| Lanolin | 2 |
| Ethoxylated fatty acid | 4 |
| CaCl₂ | 0.05 |
| Water, perfume, color | Balance |

### Example 38

### Synthesis of Non-Anionic Hybrid Copolymer Containing a Quaternary Amine Monomer and a Cationic Polysaccharide Functionality

40 grams of Nsight C-1 as a cationic starch chain transfer agent (available from AkzoNobel, Bridgewater N.J.) was initially dissolved in 100 grams of water in a reactor and heated to 98° C. A solution of 38.7 grams of dimethyl diallyl ammonium chloride (62% in water) was subsequently added to the reactor over a period of 45 minutes. An initiator solution comprising of 3.3 grams of sodium persulfate in 20 grams of water was added to the reactor at the same time as the monomer solution over a period of 45 minutes. The reaction product was held at 98° C. for an additional 60 minutes. The final product was a clear amber colored solution.

### Example 39

The performance of the non-anionic hybrid copolymer of Example 38 as an emulsion breaker is tested using the protocol detailed in Example 1 of U.S. Pat. No. 5,248,449. The synthetic oil in water emulsion is Emulsion No 3 of this example which is 75% 10W-40 Motor Oil Castrol GTX and 25% Petroleum Sulfate (Petrosul 60). The concentration of polymer of Example 37 needed to break this emulsion was around 100 to 200 ppm.

### Example 40

The performance of the non-anionic hybrid copolymer of Example 38 was tested as a flocculant. 12.5 grams of waste water with suspended particulates (bituminous tailings from an oil field in Canada) was diluted with 12.5 grams of water. 2 grams of the polymer solution of Example 38 was then added and shaken well. The suspended particulates flocced out and a clear water layer was obtained.

### Example 41

Non-anionic hybrid copolymers of Examples 32 and 33 are exemplified in the fabric softener compositions listed in Table 17.

TABLE 17
Fabric Softener composition

| Ingredient (Wt %) | Formula A | Formula B | Formula C |
|---|---|---|---|
| N,N-di(tallowoyloxyethyl)-N,N- | 10 | 14 | |
| dimethylammonium chloride. | | | |
| Methyl bis(tallow amido- | | | 2-5 |
| ethyl)2-hydroxyethyl | | | |
| ammonium methyl sulfate. | | | |
| Ethanol | 2 | 2.5 | |
| Isopropanol | | | 0.5 |
| Non-anionic hybrid copolymer | 1-10 | | |
| of Example 32 | | | |
| Non-anionic hybrid copolymer | | 1-10 | 1.5 |
| of Example 33 | | | |
| Electrolyte (calcium chloride) | 0.1-0.5 | 0.1-0.5 | 0.1-0.5 |
| Perfume | 0.1-1.5 | 0.1-1.5 | 0.1-1.5 |
| Dye, Preservative, Phase | Rest | Rest | Rest |
| Stabilizing Polymer, anti-foam | | | |
| agent, water | | | |

### Example 42

### Synthesis of Non-Anionic Hybrid Copolymer

35 grams of Hylon VII Starch (a high amylose starch containing 70% amylose) was dispersed in 132 grams of water in a reactor and heated to 52° C. The starch was depolymerized by addition of 1.07 grams of concentrated sulfuric acid (98%). The suspension was held at 52° C. for 1.5 hours. The reaction was then neutralized with 1.84 grams of 50% NaOH solution and the temperature was raised to 90° C. for 15 minutes. The starch gelatinizes and the viscosity increased during the process and a gel was formed. The viscosity dropped after the gelatinization was completed. The reaction was diluted with 30 grams of water and the temperature was lowered to 72 to 75° C. A solution of 100.1 grams of [3-(methacryloylamino)propyl]-trimethylammonium chloride (50% in water) was added to the reactor over a period of 30 minutes. An initiator solution comprising of 0.2 grams of sodium persulfate in 20 grams of water was added to the reactor at the same time as the monomer solution over a period of 35 minutes. The reaction product was held at 98° C. for an additional 2 hours. The final product was an opaque white homogenous solution.

### Example 43

### Synthesis of Non-Anionic Hybrid Copolymer

35 grams of Amioca Starch was dispersed in 88 grams of water in a reactor and heated to 52. The starch was depolymerized by addition of 0.52 grams of concentrated sulfuric acid (98%). This is half the acid used in Example 41 and causes less depolymerization of the starch resulting in a higher molecular weight. Thus the molecular weight of the polysaccharide chain transfer agent can be controlled. The suspension was held at 52° C. for 1.5 hours. The reaction was then neutralized with 0.92 grams of 50% NaOH solution and the temperature was raised to 90° C. for 15 minutes. The starch gelatinizes and the viscosity increased during the process and a gel was formed. The viscosity dropped after the gelatinization was completed. The reaction was diluted with 30 grams of water and the temperature was lowered to 72 to 75° C. A solution of 66.71 g [2-(methacryloxy)ethyl]-trimethylammonium chloride (75% in water) was added to the reactor over a period of 30 minutes. An initiator solution comprising of 0.2 grams of sodium persulfate in 20 grams of water was added to the reactor at the same time as the monomer solution over a period of 35 minutes. The reaction product was held at 98° C. for an additional 2 hours. The final product was a homogeneous opaque white paste.

### Example 44

### Clear Conditioning Shampoo Formula

A clear conditioning shampoo formula was prepared using the following ingredients:

| Ingredients | INCI Designation | % W/W | Supplier |
|---|---|---|---|
| Polymer of Example 32 | Not applicable | 0.6 | |
| Hydroxyethylurea | Not applicable | 3.00 | National |
| | | | Starch |
| Ammonium Lactate | Ammonium Lactate | 0.06 | |
| DeIonized Water | Water (Aqua) | 18.18 | |
| Standapol ES-2 | Sodium Lauryl Sulfate | 33.33 | Cognis Corp. |
| Standapol ES-3 | Sodium Laureth Sulfate | 30.00 | Cognis Corp. |
| Dehyton K | Cocamidopropyl | 10.00 | Cognis Corp. |
| | Betaine | | |
| Promodium CO | Polypropoxyethoxy- | 3.18 | Uniqema |
| | cocamide | | |
| Germaben II | Diazolidinyl Urea, | 1.00 | Sutton |
| | Propylene Glycol, | | Laboratories |
| | Methylparaben, | | |
| | Propylparaben | | |
| Sodium Chloride | Sodium Chloride | 1.00 | J. T. Baker |
| Citric Acid | Citric Acid | q.s. | |
| | | 100.00 | |

### Procedure

The ingredients are combined in the order listed above. The formulation is mixed until homogeneous after each addition.

### Example 45

### 6% VOC Root Lifting Aaerosol Mousse Formula

An exemplary 6% VOC Root lifting aerosol mousse formula was prepared using the following ingredients:

| Ingredient | INCI Designation | % W/W | Supplier |
|---|---|---|---|
| Part A | | | |
| AMAZE | Corn Starch Modified | 2.20 | National Starch |
| Polymer of Example 31 | Not applicable | 0.6 | |
| Carbopol Ultrez 10 | Acrylates/C10-30 Alkyl Acrylate | 0.05 | Noveon |
| | Crosspolymer (copolymer) | | |
| Natrosol HHR | Hydroxyethylcellulose | 0.15 | Aqualon |
| Deionized Water | Water (Aqua) | 70.32 | |
| TEA | Triethanolamine 99% | 0.05 | |
| Part B | | | |
| DC-193 | PEG-12 Dimethicone | 0.07 | Dow Corning |
| Versene 100 | Tetrasodium EDTA | 0.10 | Dow Chemical |
| Crovol Pk-70 | PEG-45 Palm Kernal Glycerides | 0.10 | Croda, Inc |
| Cropetide W | Hydrolyzed Wheat Protein (and) | 0.20 | Croda. Inc. |
| | Hydrolyzed Wheat Starch | | |
| Procetyl AWS | PPG-5 Ceteth-20 | 0.10 | Croda, Inc |
| dl-Panthenol | Panthenol | 0.10 | Ritapan |
| Rewoteric AM B-14 | Cocomidapropyl Betaine | 0.05 | Goldschmidt |
| Tween 20 | Polysorbate 20 | 0.20 | Uniqema |
| Uvinul MS-40 | Benzephenone-4 | 0.001 | BASF |
| Hydroxyethylurea | Hydroxyethyl Urea | 3.00 | National Starch |
| AmmoniumLactate | Ammonium Lactate | 0.06 | National Starch |
| Germaben II | Propylene Glycol (and) | 1.00 | Sutton Labs |
| | Diazolidinyl Urea (and) | | |
| | Methylparaben (and) | | |
| | Propylparaben | | |
| Part C | | | |
| DME | Dimethyl Ether | 6.00 | |
| Dymel 152A | Hydrofluorocarbon 152A | 16.00 | Dupont |
| | | 100.00 | |

### Procedure

The Carbopol is slowly sifted into the mixing vortex until completely dispersed. While maintaining good agitation, the NATROSOL® HHR is then slowly sifted in. Once dispersed, both the AMAZE™ and the Polymer of Example 31 is sifted in. When the solution is complete, the TEA is added. The ingredients in Part B are then added and mixed until homogeneous. Filter and fill aerosol containers. For Part C, charge with propellant.

### Example 46

### Combing Cream for Dry/Damaged Hair Formula

An exemplary 6% VOC Root lifting aerosol mousse formula was prepared using the following ingredients:

| Ingredient | INCI Designation | % W/W | Supplier |
|---|---|---|---|
| Phase A | | | |
| Cetearyl Alcohol | 30/70 Cetearyl Alcohol | 1.80 | |
| Hostacerin CS200 | Ceteareth-20 | 0.20 | Clariant |
| Genamin KDMP | Behentrimonium Chloride | 0.44 | Clariant |
| DC 949 | Amodimethicone (and) | 0.50 | Dow Corning |
| | Trideceth-12 (and) | | |
| | Cetrimonium Chloride | | |
| Phase B | | | |
| DI Water | Water (Aqua) | 88.94 | |
| STRUCTURE ZEA | Hydroxypropyl Starch | 4.00 | National Starch |
| | Phosphate | | |
| Polymer of Ex 33 | Not applicable | 1.0 | |
| Phase C | | | |
| Genamin CTAC 50 | Cetrimonium Chloride | 0.30 | Clariant |
| Phase D | | | |
| Glydant | DMDM Hydantoin | 0.20 | Lonza |
| Phenonip | Phenoxyethanol (and) | 0.15 | Nipa/Clariant |
| | Methylparaben (and) | | |
| | Ethylparaben (and) Butyl- | | |
| | paraben (and) Propylparaben | | |
| | (and) Isobutylparaben | | |
| Hydroxyethylurea | Hydroxyethylurea | 3.00 | National Starch |
| Ammonium Lactate | Ammonium Lactate | 0.06 | |
| Phase E | | | |
| Citric acid (10%) | Citric Acid | q.s. | pH 4.0-5.0 |
| | | 100.00 | |

### Procedure

Dissolve STRUCTURE ZEA into the water at room temperature. Add the Polymer of Example 33 and heat to 80° C. while mixing (Phase B). In a separate vessel, combine Phase A and heat to 80° C. Add Phase B to Phase A with agitation. Add Phase C while maintaining temperature (80° C.). Continue mixing and cool to 45° C. Add Phase D and adjust pH, if necessary.

### Example 47

### Conditioning Styling Gel Formula

An exemplary conditioning styling gel formula was prepared using the following ingredients:

| Ingredient | INCI Designation | % W/W | Supplier |
|---|---|---|---|
| Part A | | | |
| Deionized Water | Water (Aqua) | 50.00 | |
| AMAZE XT | Dehydroxanthan Gum | 1.00 | National Starch |
| Part B | | | |
| Deionized Water | Water (Aqua) | 41.74 | |
| Polymer of Ex 38 | Not applicable | 0.3 | |
| Part C | | | |
| Propylene Glycol | Propylene Glycol | 2.00 | |
| DL-Panthenol | Panthenol | 0.50 | Roche |
| Na2EDTA | Disodium EDTA | 0.05 | |
| Hydroxyethylure a | Hydroxyethylurea | 3.00 | |
| Ammonium | Ammonium Lactate | 0.06 | |
| Lactate | | | |
| Cropeptide W | Hydrolyzed Wheat | 1.00 | Croda |
| | Protein and Hydrolyzed | | |
| | Wheat Starch | | |
| DC 193 | PEG-12 Dimethicone | 0.20 | Dow Corning |
| Glydant Plus | DMDM Hydantoin and | 0.30 | |
| Granular | Iodopropynyl Butyl- | | |
| | carbamate | | |
| | | 100.00 | Lonza |

### Procedure

Dust AMAZE XT into the water in Part A and mix until completely hydrated. Separately, combine the ingredients of Part B and mix until dissolved. Add Part B to Part A with agitation. Add remaining ingredients and mix until uniform.

### Example 48

### Leave-in Conditioner Formula

An exemplary leave-in conditioner formula was prepared using the following ingredients:

| Ingredients | INCI Designation | % W/W | Supplier |
|---|---|---|---|
| Phase A | | | |
| Polymer of Ex 42 | Not applicable | 1.2 | |
| Deionized Water | Water (Aqua) | 48.00 | |
| dl-Panthenol | Panthenol | 0.50 | Tri-K |
| | | | Industries |
| Phase B | | | |
| Deionized Water | Water (Aqua) | 44.79 | |
| TEA | Triethanolamine | 0.20 | |
| Neo Heliopan, Phenyl | Benzimidazole Sulfonic | 0.20 | Haarmann |
| Type Hydro | Acid | | & Reimer |
| DC 929 Cationic | Amodimethicone (and) | 0.75 | Dow |
| Emulsion | Tallowtrimonium Chloride | | Corning |
| | (and) Nonoxynol-10 | | |
| Phase C | | | |
| Solu-Silk Protein | Hydrolyzed Silk | 1.00 | Brooks |
| | | | Industries |
| Versene 100 | Tetra Sodium EDTA | 0.20 | Dow |
| | | | Chemical |
| Glydant | DMDM Hydantoin | 1.00 | Lonza |
| Hydroxyethylurea | Hydroxyethylurea | 3.00 | |
| Ammonium Lactate | Ammonium Lactate | 0.06 | |
| Fragrance | Fragrance (Perfume) | q.s. | |
| | | 100.00 | |

### Prepartation

Prepare Phase A by dissolving the Polymer of Example 42 in water using good agitation. Mix until solution is clear and homogenous. Add dl-Panthenol and allow to completely dissolve. Prepare Phase B by adding TEA to water and mix well. Add Neo Heliopan and mix until clear. Follow with DC 929 cationic emulsion. Combine parts by adding Phase B to Phase A. Mix well and continue to mix for approximately 15 minutes. Add Solu-silk and mix well. Add Versene 100, Glydant, hydroxyethylurea, ammonium lactate, and fragrance, mixing well after each addition.

### Example 49

### Clear Conditioner with Suspended Beads

An exemplary clear conditioner with suspended beads was prepared using the following ingredients:

| Ingredients | INCI Designation | % W/W | Supplier |
|---|---|---|---|
| Phase A | | | |
| Deionized Water | Water (Aqua) | 78.74 | |
| Polymer of Ex 43 | Not applicable | 1.0 | |
| Glydant | DMDM Hydantoin | 0.50 | Lonza |
| Propylene Glycol | Propylene Glycol | 2.00 | |
| Arquad 16-25W | Cetrimonium Chloride | 2.00 | Akzo-Nobel |
| STRUCTURE PLUS | Acrylates/Amino- | 10.00 | National |
| | acrylates/C10-30 Alkyl | | Starch |
| | PEG-20 Itaconate | | |
| | Copolymer | | |
| Hydroxyethylurea | Hydroxyethylurea | 3.00 | |
| Ammonium | Ammonium Lactate | 0.06 | |
| Lactate | | | |
| Versene 100 | Tetrasodium EDTA | 0.05 | Dow |
| | | | Chemical |
| Phase B | | | |
| Silsoft A-858 | Dimethicone Copolyol | 2.00 | CK Witco OSI |
| | Bishydroxyethylamine | | |
| Neo Heliopan AV | Ethylhexyl | 0.05 | Haarman |
| | Methoxycinnamate | | & Reimer |
| Phase C | | | |
| Glycolic Acid (70%) | Glycolic Acid | 0.45 | |
| Phase D | | | |
| Florabeads | Jojoba Esters | 0.80 | Floratech |
| | | 100.00 | |

### Procedure

Polyquaternium-4 is dissolved in water with mixing. The remaining ingredients of Phase A are sequentially added with continued mixing. Phase B is combined and then added to Phase A. Continue to mix while slowly adding glycolic acid to Phase AB, taking care to avoid entrapped air. Finally, add beads slowly while mixing.

### Example 50

### 55% VOC Firm Hold, Crystal Clear Pump Hairspray Formula

An exemplary 55% VOC firm hold, crystal clear pump hairspray formula was prepared using the following ingredients:

| Ingredients | INCI Designation | % W/W | Supplier |
|---|---|---|---|
| Polymer of Ex 24 | Not applicable | 12.00 | |
| AMP (reg) | Aminomethyl Propanol | 0.85 | Dow Chemical |
| Deionized Water | Water (Aqua) | 29.09 | |
| Hydroxyethylurea | Hydroxyethylurea | 3.00 | |
| Ammonium Lactate | Ammonium Lactate | 0.06 | |
| *SD Alcohol 40 | SD Alcohol 40 | 55.00 | |
| | | 100.00 | |

### Preparation

Dissolve AMP in SD Alcohol 40 and water. While maintaining proper agitation, slowly pour in BALANCE 0/55. Add remaining ingredients and mix until homogenous.

### Example 51

### Sunscreen Formulas

Exemplary sunscreen formulas were prepared using the following ingredients:

| Ingredient | Function | Formula A | Formula B | Formula C |
|---|---|---|---|---|
| PHASE A | | | | |
| Isohexadecane | Emollient | 1.5 | 1.5 | 1.5 |
| C12-C15 alkyl benzoate | Emollient | 3.0 | 3.0 | 3.0 |
| Cyclopentasiloxane | Emollient | 2.25 | 2.25 | 2.25 |
| Sorbitan Stearate | Emulsifier | 1.0 | 1.0 | 1.0 |
| Glyceryl Stearate | Emulsifier | 2.0 | 2.0 | 2.0 |
| (and) PEG-100 Stearate | | | | |
| Caprylic/Capric Triglyceride | Solubilizer | 0.0 | 6.25 | 6.25 |
| Isopropyl Myristate | Solubilizer | 0.0 | 6.25 | 6.25 |
| Octocrylene | UVB filter (org) | 2.0 | 0.0 | 0.0 |
| Ethylhexyl Methoxycinnamate | UVB filter (org) | 7.5 | 0.0 | 0.0 |
| Benzophenone-3 | UVB filter (org) | 3.0 | 0.0 | 0.0 |
| ZnO (and) C12-C15 Alkyl Benzoate | UVA/B filter (inorg) | 0.0 | 6.0 | 6.0 |
| (and) Polyhydroxystearic Acid | | | | |
| PHASE B | | | | |
| Water | | 67.25 | 54.25 | 58.65 |
| Dehydroxanthan Gum | Suspension agent, | 0.5 | 0.5 | 0.0 |
| | Rheology modifier | | | |
| Xanthan Gum | Rheology modifier | 0.0 | 0.0 | 0.5 |
| Polymer of Example 20 | Film former | 4.4 | 4.4 | 4.4 |
| Glycerin | Humectant | 3.0 | 3.0 | 3.0 |
| TiO₂ and Alumina and Silica | UVB filter (inorg) | 0.0 | 7.0 | 7.0 |
| and Sodium Polyacrylate | | | | |
| PHASE C | | | | |
| Corn Starch Modified | Aesthetic enhancer | 2.0 | 2.0 | 2.0 |
| DMDM Hydantoin and | Preservative | 0.6 | 0.6 | 0.6 |
| Iodopropyl Butylcarbamate | | | | |
| Citric Acid (50%) | Neutralizer | qs to pH 7 | qs to pH 7 | qs to pH 7 |
| TOTAL | | 100 | 100 | 100 |

### Example 52

### Synthesis of Non-Ionic Hybrid Copolymer with Polysaccharide Chain Transfer Agent

Hydroxyethyl cellulose (QP 300 available from Dow) was depolymerized in the following manner. Thirty grams of QP 300 was introduced in to 270 g of deionized water with stirring. 0.05 g of Ferrous ammonium sulfate hexahydrate and 1 g of hydrogen peroxide (H₂O₂) solution (35% active) was added. The mixture was heated to 60° C. and held at that temperature for 30 minutes. This depolymerized CMC solution was then heated to 90° C.

A solution of 38.7 grams of dimethyl diallyl ammonium chloride (62% in water) is subsequently added to the reactor over a period of 50 minutes. An initiator solution comprising of 2 grams of V-086 2,2′-Azobis [2-methyl-N-(2-hydroxyethyl) propionamide] azo initiator from Wako Pure Chemical Industries, Ltd., Richmond, Va.] in 30 grams of water is added to the reactor at the same time as the monomer solution over a period of 60 minutes. The reaction product is held at 90° C. for an additional 60 minutes.

### Example 53

### Synthesis of Anionic Hybrid Copolymer with Lignosulfonate as a Naturally Derived Hydroxyl Containing Chain Transfer Agent

150 grams of a naturally derived hydroxyl containing chain transfer agent a lignosulfonate (ARBO S08 50% solution available from Tembec Chemical Products Group) was heated to 75° C. A monomer solution containing 100 grams of acrylic acid was subsequently added to the reactor over a period of 50 minutes. An initiator solution comprising of 2 grams of V-50 [2,2′-azobis(2-amidinopropane) dihydrochloride] azo initiator from Wako Pure Chemical Industries, Ltd., Richmond, Virginia] in 30 grams of water was added to the reactor at the same time as the monomer solution over a period of 60 minutes. The reaction product was held at 75° C. for an additional 60 minutes. The reaction product was neutralized with 100 grams of a 50% solution of NaOH and diluted with 200 grams of water. The final product was a dark amber/black colored solution.

### Example 54

### Synthesis of Anionic Hybrid Copolymer with Lignosulfonate as a Naturally Derived Hydroxyl Containing Chain Transfer Agent

125 grams of a naturally derived hydroxyl containing chain transfer agent a lignosulfonate (ARBO S08 50% solution available from Tembec Chemical Products Group) was mixed with 50 grams of water and heated to 85° C. A monomer solution containing 15 grams of acrylic acid and 50 grams of a 50% solution of Na 2-acrylamido-2-methyl propane sulfonate was subsequently added to the reactor over a period of 60 minutes. An initiator solution comprising of 2 grams sodium persulfate in 40 grams of water was added to the reactor at the same time as the monomer solution over a period of 60 minutes. The reaction product was held at 85° C. for an additional 60 minutes. The reaction product was neutralized with 8 grams of a 50% solution of NaOH. The final product was a dark amber/black colored solution.

### Example 55

### Synthesis of Ester Hybrid Copolymer Containing an Ester Monomer Derived from a Dicarboxylic Acid

102 grams of monomethylmaleate (ester monomer) was dissolved in 150 grams of water.

5.4 grams of ammonium hydroxide was added and the mixture was heated to 87C. 340 grams of a naturally derived hydroxyl containing chain transfer agent a lignosulfonate (ARBO S08 50% solution available from Tembec Chemical Products Group) was added to the reactor. A monomer solution containing a mixture of 64.6 grams of acrylic acid and 3.4 grams of hydroxypropyl methacrylate was added to the reactor over a period of 5 hours. A first initiator solution comprising of 21 grams of erythorbic acid dissolved in 99 grams of water was added over a period of 5.5 hours. A second initiator solution comprising of 21 grams of a 70% solution of tertiary butyl hydroperoxide dissolved in 109 grams of water was added over a period of 5.5 hours. The reaction product was held at 87° C. for 60 minutes. The final product was a dark amber/black colored solution.

### Example 56

### Synthesis of Ester Hybrid Copolymer Containing an Ester Monomer Derived from a Dicarboxylic Acid

32.9 grams of maleic anhydride was dissolved in 140 grams of water. 318 grams of a naturally derived hydroxyl containing chain transfer agent a lignosulfonate (ARBO S08 50% solution available from Tembec Chemical Products Group) was added to the reactor. 33.4 grams of 50% sodium hydroxide solution was added and the mixture was heated to 87C. 0.015 grams of ferrous ammonium sulfate hexahydrate dissolved in 6 grams of water was then added to the reactor. A monomer solution containing a mixture of 49.3 grams of acrylic acid in 6.4 grams of water was added to the reactor over a period of 4 hours. An initiator solution comprising of 28.9 grams of 35% hydrogen peroxide solution and 3.6 grams of sodium persulfate dissolved in 14 grams of water was added over a period of 4 hours. The reaction product was held at 87° C. for 60 minutes. The final product was a dark amber colored solution.

### Example 57

The polymer of Example 55 was tested in a phosphate inhibition test described in Example 1 of U.S. Pat. No. 5,547,612.

TABLE 18

| | Polymer | % |
|---|---|---|
| | concentration | phosphate |
| Polymer | (ppm) | inhibition |
| Example 55 | 25 | 11 |
| Example 55 | 50 | 95 |
| Example 55 | 100 | 100 |

Calcium phosphate inhibition numbers above 80% are considered to be acceptable in this test. These data in Table 18 above indicate that the polymers of this invention are excellent calcium phosphate inhibitors.

### Example 58

The polymer of Example 56 was tested in a carbonate inhibition test described in Example 8 of U.S. Pat. No. 5,547,612.

TABLE 19

| | Polymer | % |
|---|---|---|
| | concentration | phosphate |
| Polymer | (ppm) | inhibition |
| Example 55 | 25 | 81 |

Calcium carbonate inhibition numbers above 80% are considered to be acceptable in this test. These data in Table 19 above indicate that the polymers of this invention are excellent calcium carbonate inhibitors.

### Example 59

A reactor containing 198.0 grams of maltodextrin as a polysaccharide transfer agent (Star Dri 180, DE 18 spray-dried maltodextrin available from Tate & Lyle, Decatur, Ill.) dissolved in 200.0 grams of water was heated to 95° C. A monomer solution containing 2.0 grams of acrylic acid (0.028 moles) and 70.0 grams of water was added to the reactor over a period of 1 hour. An initiator solution containing 0.10 grams of sodium persulfate and 30.0 grams of water was added simultaneously over a period of 1 hour and 10 minutes. The reaction product was held at 95° C. for an additional 1 hour. The polymer was then neutralized by adding 1.0 grams of a 50% sodium hydroxide solution. The final product was a clear, light amber colored solution.

### Example 60

A reactor containing 199.0 grams of maltodextrin as a polysaccharide transfer agent (Star Dri 180, DE 18 spray-dried maltodextrin available from Tate & Lyle, Decatur, Ill.) dissolved in 200.0 grams of water was heated to 95° C. A monomer solution containing 1.0 grams of acrylic acid (0.014 moles) and 60.0 grams of water was added to the reactor over a period of 1 hour. An initiator solution containing 0.05 grams of sodium persulfate and 30.0 grams of water was added simultaneously over a period of 1 hour and 10 minutes. The reaction product was held at 95° C. for an additional 1 hour. The polymer was then neutralized by adding 0.5 grams of a 50% sodium hydroxide solution. The final product was a clear, light amber colored solution.

### Example 61

A reactor containing 75.0 grams of water and 27.8 grams of a 50% sodium hydroxide solution was heated to 100° C. A solution containing 50.0 grams of acrylic acid, 25.0 grams maltodextrin as a polysaccharide transfer agent (Star Dri 100, DE 10 spray-dried maltodextrin available from Tate & Lyle, Decatur, Ill.) and 60.0 grams of water was added to the reactor over a period of 45 minutes. An initiator solution containing 3.3 grams of sodium persulfate and 28.0 grams of water was added simultaneously over a period of 1 hour. The reaction product was held at 100° C. for an additional 1 hour. The solution was a clear amber color with no crosslinking. This illustrates that crosslinking can be eliminated by reducing the reactivity of the monomer. In this case the monomer activity was reduced by the addition of the 50% NaOH.

### Example 62

A reactor containing 75.0 grams of water and 18.6 grams of a 50% sodium hydroxide solution was heated to 100° C. A solution containing 33.5 grams of acrylic acid, 41.5 grams maltodextrin as a polysaccharide transfer agent (Star Dri 100, DE 10 spray-dried maltodextrin available from Tate & Lyle, Decatur, Ill.) and 60.0 grams of water was added to the reactor over a period of 45 minutes. An initiator solution containing 3.3 grams of sodium persulfate and 28.0 grams of water was added simultaneously over a period of 1 hour. The reaction product was held at 100° C. for an additional 1 hour. The solution was a clear amber color with no crosslinking. This illustrates that crosslinking can be eliminated by reducing the reactivity of the monomer. In this case the monomer activity was reduced by the addition of the 50% NaOH.

### Example 63

A series of dispersancy tests to prove that hybrid polymers containing greater than 75 weight percent chain transfer agent have unexpected performance benefits. Maltodextrin was used as a chain transfer agent. A series of polymers were synthesized containing 33%, 55%, 80%, 85%, 90%, 95%, 99% and 99.5 maltodextrin and the process to make these samples are described in Examples 60, 61, 1, 3, 5, 2, 58 and 59 respectively.

The dispersancy test was performed as follows: The samples were prepared by adding 2% clay (50:50 rose clay:spinks black clay) to deionized water. The samples were then stirred on a magnetic stir plate for 20 minutes, after which 0.1% active dispersant was added and the samples were stirred for one minute more. The suspensions were then poured into 100 ml graduated cylinders and allowed to rest. The amount of clear supernatant liquid on the top of the cylinders was measured after 1 hour and after 24 hours. The lower the amount of the clear supernatant liquid the better the dispersancy performance.

The data in FIG. 1 (1 hour) and FIG. 2 (24 hours) indicate that a typical maltodextrin (Star Dri 180, DE 18 spray-dried maltodextrin available from Tate & Lyle, Decatur, Ill.) chain transfer agent by itself does not have any dispersancy performance. FIG. 3 also illustrates the results after a 1 hour dispersancy test between samples of a hybrid copolymer having at least one anionic ethylenically unsaturated monomer shown with maltodextrin present in amounts of 55%, 80%, 95%, 99% and 99.5%, compared to samples of a polyacrylate (AR4) having 0% maltodextrin present and a sample having 100% maltodextrin (MD) present. A commercial polyacrylate Aquatreat® AR-4 available from AkzoNobel Surface Chemistry performs very well. Therefore, as the weight percent of the chain transfer agent in the hybrid polymer increases, the dispersancy performance of the polymer should decrease (see dotted line in FIGS. 1 and 2). Surprisingly, it has been found that anionic hybrid polymers containing greater than 75 weight percent maltodextrin as a chain transfer agent have good dispersancy performance even though a drop in performance is expected based on the performance of the polyacrylate and the maltodextrin by itself. The performance of these copolymers are similar to those containing 33 and 55 weight percent maltodextrin of US Publication No. 20070021577(A1). Even more surprisingly the sample containing 99 weight percent maltodextrin has very good dispersancy performance and the performance of the hybrid copolymers starts to drop at the 99.5% chain transfer level (FIG. 2). Clearly the polymers of this invention containing greater than 75 weight percent chain transfer agent have an unexpected benefit.

All documents cited in the Detailed Description of the Invention are, in relevant part, incorporated herein by reference; the citation of any document is not to be construed as an admission that it is prior art with respect to the present invention.

While particular embodiments of the present invention have been illustrated and described herein, the invention is not intended to be limited to the details shown. Rather, various modifications may be made in the details within the range and scope of equivalents of the claims and without departing from the spirit and scope of the invention.

What is claimed:

1. A hybrid copolymer comprising:
a synthetic polymer derived from at least one anionic ethylenically unsaturated monomer and a naturally derived hydroxyl containing chain transfer agent as an end group, wherein the chain transfer agent is present from about 75% by weight to about 99% by weight, based on the total weight of the hybrid copolymer.

2. The copolymer of claim 1 wherein the chain transfer agent is selected from the group consisting of cellulose, derivatives of cellulose, inulin, and derivatives of inulin.

3. The copolymer of claim 1 wherein the chain transfer agent is lignin or a derivative of lignin.

4. The copolymer of claim 1 wherein the copolymer is water soluble.

5. The copolymer of claim 1 wherein the copolymer has an average molecular weight of less than about 500,000.

6. The copolymer of claim 1 wherein the chain transfer agent has a molecular weight of less than about 500,000.

7. The copolymer of claim 1 wherein the chain transfer agent is selected from the group consisting of glycerol, citric acid, lactic acid, tartaric acid, gluconic acid, ascorbic acid and glucoheptonic acid.

8. The copolymer of claim 1 wherein the chain transfer agent is a saccharide or a saccharide derivative.

9. The copolymer of claim 1 wherein the copolymer has an absorption in a range of from about 300 to about 800 nanometers when activated.

10. A composition comprising the copolymer of claim 1, wherein the composition is selected from the group consisting of a cleaning composition, a superabsorbent composition, a fiberglass binder composition, a rheology modifier composition, an oil field composition, a water treatment composition, a dispersant composition, a cement composition and a concrete composition.

11. The composition of claim 10 wherein the composition is the cleaning composition and said cleaning composition is a detergent, fabric cleaner, automatic dishwashing detergent, glass cleaner, rinse aids, fabric care formulation, fabric softener, flocculants, coagulants and emulsion breakers, hard surface cleaner or a laundry detergent.

12. The composition of claim 11 wherein composition is the automatic dishwashing detergent and said automatic dishwashing detergent is substantially free of phosphates.

13. The composition of claim 12 further comprising alkali metal or alkali-metal earth carbonates, citrates or silicates.

14. The composition of claim 11 further comprising low foaming non-ionic surfactants.

15. The composition of claim 11 further comprising glutamic acid N,N-diacetic acid (GLDA) or methylglycine N,N-diacetic acid (MGDA).

16. A hybrid copolymer comprising:
a synthetic polymer derived from at least one of a non-anionic ethylenically unsaturated monomer and a naturally derived hydroxyl containing chain transfer agent as an end group.

17. The copolymer of claim 16 wherein said ethylenically unsaturated monomer is cationic or nonionic.

18. The copolymer of claim 16 wherein the copolymer is water soluble.

19. A composition comprising the non-anionic hybrid copolymer of claim 16 and an adjunct ingredient wherein the adjunct ingredient is selected from the group consisting of fabric softener actives, water, surfactants, electrolyte, phase stabilizing polymers, silicones, perfume, nonionic surfactant, non-aqueous solvent, fatty acid, dye, preservatives, optical brighteners and antifoam agents.

20. The composition according to claim 19 wherein the composition is selected from the group consisting of a fabric care formulation, fabric softener, flocculants, coagulants and emulsion breakers.

21. The copolymer of claim 19 wherein the copolymer has an absorption in a range of from about 300 to about 800 nanometers when activated with sulfuric acid and phenol.

22. A hybrid copolymer comprising:
a synthetic portion derived from at least one ester monomer and a naturally derived hydroxyl containing chain transfer agent as an end group.

23. The copolymer of claim 22 wherein the at least one ester monomer is derived from a dicarboxylic acid.

24. The copolymer of claim 23 wherein the at least one ester monomer is selected from the group consisting of monomethylmaleate, dimethylmaleate, monomethylitaconate, dimethylitaconate, monoethylmaleate, diethylmaleate, monoethylitaconate, diethylitaconate, monobutylmaleate, dibutylmaleate, monobutylitaconate and dibutylitaconate.

25. The copolymer of claim 22 wherein the synthetic portion further contains an ethylenically unsaturated monomer.

26. An ester graft copolymer comprising:
a naturally derived hydroxyl moiety backbone and side chains derived from at least one ester monomer.

27. The copolymer of claim 26 wherein the ester monomer is derived from dicarboxylic acid.

28. The copolymer of claim 27 wherein the at least one ester monomer is selected from the group consisting of monomethylmaleate, dimethylmaleate, monomethylitaconate, dimethylitaconate, monoethylmaleate, diethylmaleate, monoethylitaconate, diethylitaconate, monobutylmaleate, dibutylmaleate, monobutylitaconate and dibutylitaconate.

29. The ester graft copolymer of claim 26 wherein the synthetic portion further contains an ethylenically unsaturated monomer.

30. A composition comprising:
the hybrid copolymer of claim 22 and at least one adjunct ingredient.

31. A method of determining the concentration of a hybrid copolymer in an aqueous system, the method comprising:
reacting a sample of an aqueous hybrid copolymer comprising a synthetic polymer derived from at least one anionic ethylenically unsaturated monomer and a naturally derived hydroxyl containing chain transfer agent as the end group with an effective amount of photoactivator under conditions effective to cause the hybrid copolymer to absorb with the wavelength in the range of from 300 to 800 nanometers;
measuring the absorbance of the aqueous sample and comparing the absorbance of the aqueous sample to a predetermined calibration curve of known absorbances and concentrations; and
comparing the absorbance of the aqueous sample to the known concentrations and known absorbences to determinine the concentration of the hybrid copolymer.

32. A method of preparing a hybrid copolymer comprising polymerizing at least one monomer with a solution of a naturally derived hydroxyl containing chain transfer agent having a minor amount of secondary chain transfer agents.

33. The method of claim 32 further comprising catalyzing said polymerizing step with an initiator that is substantially free of a metal ion initiating system at a temperature sufficient to activate said initiator.

34. A blend comprising:
a hybrid copolymer comprising a synthetic polymer derived from at least one anionic ethylenically unsaturated monomer and a naturally derived hydroxyl containing chain transfer agent as an end group; and
a builder or a chelating agent selected from the group consisting of alkali metal or alkali-metal earth carbonates, alkali metal or alkali earth citrates, alkali metal or alkali earth silicates, glutamic acid N,N-diacetic acid (GLDA), methylglycine N,N-diacetic acid (MGDA) and combinations thereof.

35. The blend of claim 34, wherein the blend is a particulate containing a uniform dispersion of the hybrid copolymer and the builder or chelating agent, and the particulate is a powder or a granule.

36. The blend of claim 34 wherein the chain transfer agent is present from about 75% by weight to about 99% by weight, based on the total weight of the hybrid copolymer.
